(19) United States
(12) Patent Application Publication
(10) Pub. No.: US 2021/0079068 A1
(43) Pub. Date: Mar. 18, 2021

(54) ANTIBODY GENE THERAPY FOR TREATMENT AND PREVENTION OF INFECTION BY RABIES LYSSAVIRUS

(71) Applicants: Auburn University, Auburn, AL (US); University of Massachusetts Medical School, Worcester, MA (US)

(72) Inventors: Henry J. Baker, Auburn, AL (US); Douglas R. Martin, Auburn, AL (US); Miguel Sena-Esteves, Worcester, MA (US); Kent R. Van Kampen, Payson, UT (US)

(73) Assignees: Auburn University, Auburn, AL (US); University of Massachusetts Medical School, Worcester, MA (US)

(21) Appl. No.: 17/024,424

(22) Filed: Sep. 17, 2020

Related U.S. Application Data
(60) Provisional application No. 62/901,426, filed on Sep. 17, 2019.

(51) Int. Cl. C07K 16/10; A61K 48/00; C12N 15/86; A61P 31/14

(52) U.S. Cl. CPC C07K 16/10; A61K 48/0058; C12N 15/86; C07K 2317/21; C07K 2317/76; C12N 2750/14143; C12N 2830/008; A61P 31/14

(57) ABSTRACT

Disclosed are compositions, vectors, and methods for treating and preventing rabies lyssavirus infection in a subject in need thereof, including rabies lyssavirus encephalitis. The disclosed compositions relate to anti-rabies immunoglobulins and vectors for expressing anti-rabies immunoglobulins such as adeno-associated virus (AAV) vectors that express anti-rabies immunoglobulins in a subject in need thereof. In some embodiments, the disclosed methods relate to treating and/or preventing an infection by rabies lyssavirus in a subject in need thereof, the methods comprising administering to the subject a dose of an adeno-associated virus (AAV) vector that expresses an immunoglobulin that binds and neutralizes rabies lyssavirus in the subject.

### CROSS-REFERENCE TO RELATED APPLICATIONS

This application claims the benefit under 35 U.S.C. § 119(e) to U.S. Provisional Application No. 62/901,426, filed on Sep. 17, 2019, the content of which is incorporated herein by reference in its entirety.

### INCORPORATION-BY-REFERENCE OF MATERIAL SUBMITTED IN ELECTRONIC FORM

A Sequence Listing accompanies this application and is submitted as an ASCII text file of the sequence listing named “169996_00468_ST25.txt” which is 13.0 KB in size and was created on Sep. 17, 2020. The sequence listing is electronically submitted via EFS-Web with the application and is incorporated herein by reference in its entirety.

### BACKGROUND

The present invention relates generally to compositions, vectors, and methods for treating and preventing infections by rabies lyssavirus in a subject in need thereof. In particular, the invention relates to recombinant adeno-associated virus (AAV) vectors that express neutralizing immunoglobulins in the subject in the nervous system and other tissues and the use thereof for treating and preventing infections by rabies lyssavirus, including rabies lyssavirus encephalitis.

Rabies virus is a neurotropic lyssavirus that causes fatal, untreatable encephalitis. Three billion people live in 150 countries where rabies is enzootic and an estimated 20 million people are exposed to rabies infection each year. Rabies is preventable, but as many as 60 thousand people, 40-60% of them children, die each year of fatal and incurable rabies encephalitis in less developed countries where rabies is enzootic in dogs. Each year, 15 million patients receive post-exposure treatment and although it can be lifesaving, post-exposure therapy is underutilized because multiple treatments are required to achieve protective antibody titers, intensive medical care is often required and the treatment is costly. Post-exposure antibody gene therapy could greatly simplify rabies treatment and save lives, even in areas where current heroic measures are beyond medical standards of care (Rabies vaccines: WHO position paper, 2010).

The surface of rabies virus particles is covered with thumb-like envelope glycoproteins (rabies virus G protein, or RVG) that mediate cell attachment and entry (FIG. 7). The RVG protein is highly immunogenic and is the antigen used in vaccines (Franka, et al 2013). Current vaccines include a killed virus vaccine similar to those developed by Pasteur hundreds of years ago, which is effective only if multiple doses are given initially and repeated to maintain protection. These vaccines also carry risks of adverse responses to formulations that include anaphylactic antigens, preservatives and toxic chemicals. In high-risk patients, pre-exposure antibody titers are measured every 12 to 24 months and re-immunization is administered when titers fall below protective values. While such a rigorous, time consuming, sophisticated and expensive laboratory support may be feasible for patients in the developed world, it is not a practical approach for people in the poorest countries, where rabies exposure is greatest. A recombinant pox-virus vectored rabies vaccine is licensed for animals, but it must be repeated every 3 years. Antibody gene therapy for rabies provides a solution for human and animal rabies prevention and treatment.

### SUMMARY

Disclosed are compositions, vectors, and methods for treating and preventing rabies lyssavirus infection in a subject in need thereof, including rabies lyssavirus encephalitis. The disclosed compositions relate to anti-rabies immunoglobulins and vectors for expressing anti-rabies immunoglobulins such as adeno-associated virus (AAV) vectors that express anti-rabies immunoglobulins in the subject. In some embodiments, the disclosed methods relate to treating and/or preventing an infection by rabies lyssavirus in a subject in need thereof, the methods comprising administering to the subject a dose of an adeno-associated virus (AAV) vector that expresses an immunoglobulin in the nervous system and other tissues that binds and neutralizes rabies lyssavirus in the subject.

### BRIEF DESCRIPTION OF THE DRAWINGS

FIG. 1. Map of adeno-associated virus vector for expressing rabies antibody (AAV-RAB) (plasmid pAAV-CBA-CR57-nHL-wpre Clone 2969).

FIG. 2. Gene sequence of anti-rabies antibody gene pAAV-CBA-CR57-nHL-wpre Clone 2969 (SEQ ID NO:1).

FIG. 3. Gene sequence of anti-rabies antibody gene pAAV-CBA-CR57v1.1-wpre Clone 2970_2 (SEQ ID NO:2).

FIG. 4. Gene sequence of anti-rabies antibody gene pAAV-CBA-CR57v3.1-wpre Clone 2971 (SEQ ID NO:3).

FIG. 5. Exemplary AAV vector expressing a broadly neutralizing antibody to the rabies virus comprising: ITR, inverted terminal repeats; CBA, regulatory element consisting of cytomegalovirus enhancer and chicken (3-actin promoter; H+L, heavy and light chain cDNAs; wpre, woodchuck hepatitis virus post-transcriptional regulatory element; pA, poly-adenylation signal.

FIG. 6. Types of Antibody fragments of naturally occurring and synthetic antibodies. Top row: F(ab′)2 fragment, Fab′ fragment, single-chain variable fragment, di-scFv. Bottom row: trifunctional antibody, chemically linked F(ab′)2, bi-specific T-cell engager.

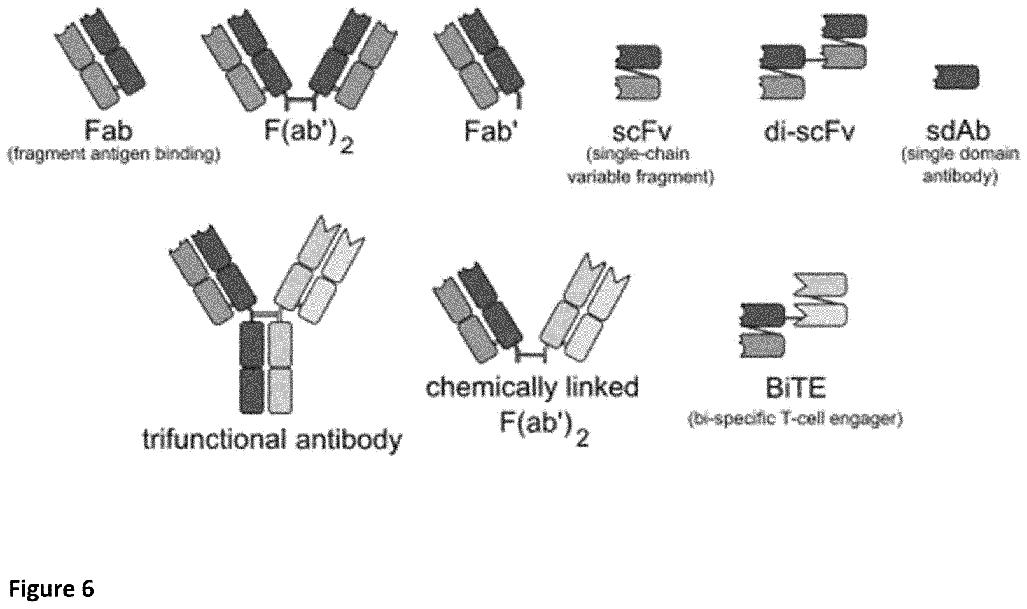

FIG. 7. Graphic representation of a rabies virus particle showing Glycoprotein G surface projections from the envelope and the internal reproductive proteins and genome.

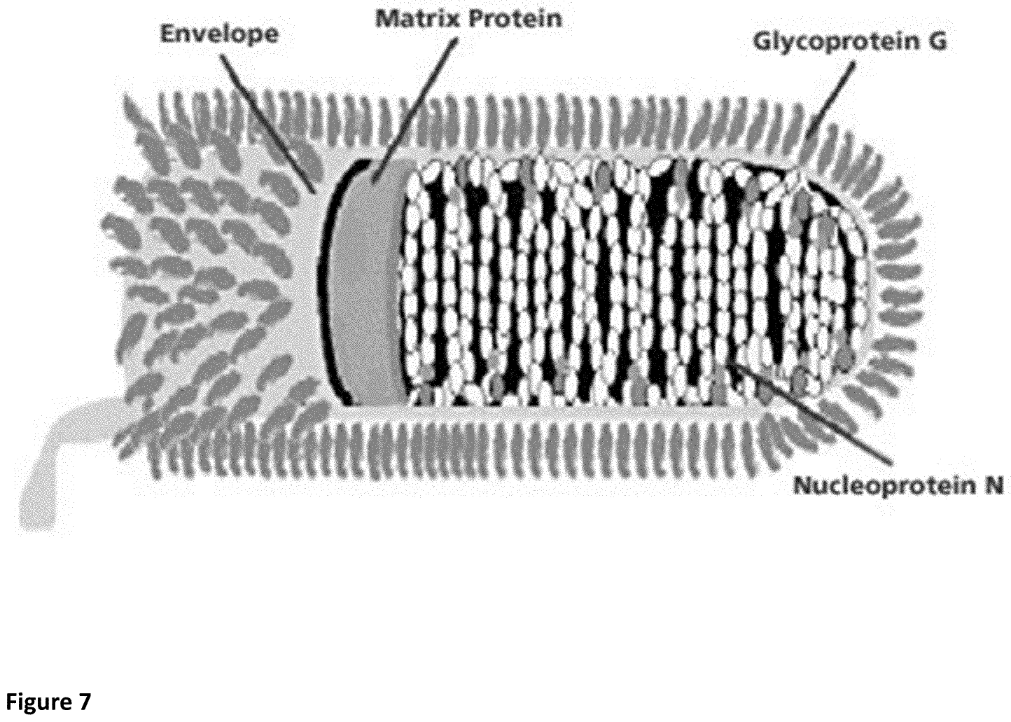

FIG. 8. Exemplary amino acid sequence of expression cassette for rabies virus antibody gene (SEQ ID NO:4): Human Growth Hormone Peptide Signal (aa 1-26); Rabies VH (aa 27-151); Mouse IgG1 heavy chain constant region (aa 152-475); 2A Sequence Mediating Ribosome Skipping (aa 476-507); Human Growth Hormone Peptide Signal (aa 508-533); Rabies VL (aa 534-649); Mouse Kappa Light Chain Constant Region (650-755).

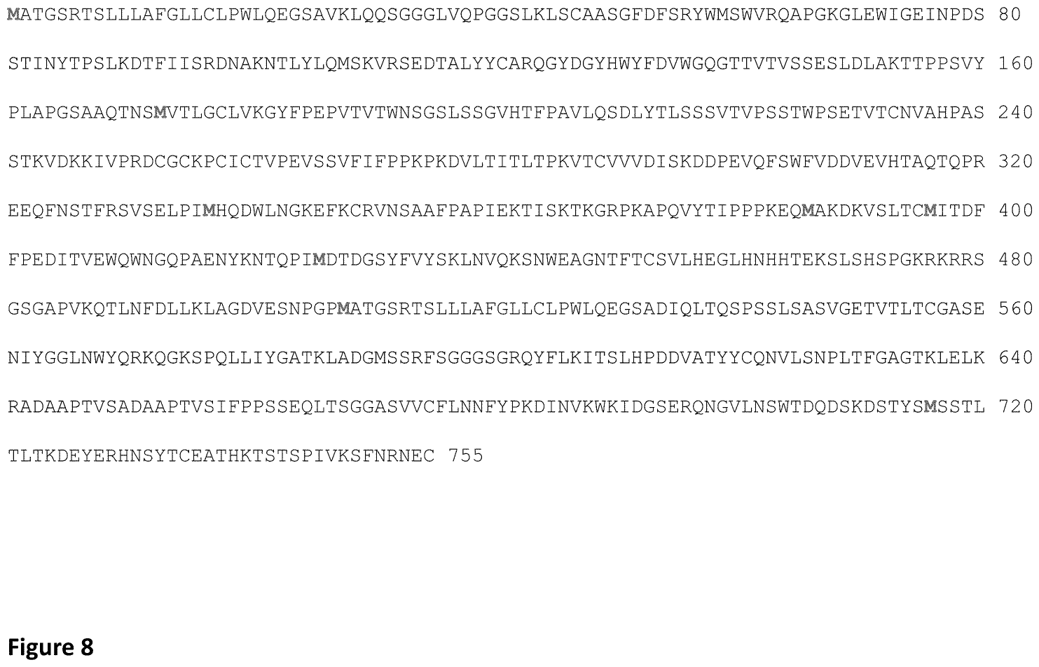

FIG. 9. Rabies virus neutralization assays from serum of treated mice. C57BL/6J mice were treated by intravenous injection of an AAV9 vector expressing the human CR57 broadly neutralizing antibody to rabies virus. Mice were treated with one of two doses (6×10¹³ or 1.5×10¹³ vg/kg body weight). Serum was collected at 2-3, 4 and 8 weeks post-injection (n=7-8 mice per time point). Neutralizing titers were converted to International Units/ml (IU/ml), and 0.5 IU/ml is considered protective in humans. Untreated controls (n=6) had titers below the limit of detection (<2.8 IU/ml).

FIG. 10. An ELISA plate was coated with a commercial rabies vaccine. Brain homogenates were diluted to various concentrations (ng/μ1) and added to the ELISA plate (100 μl). Each treated and control sample was run in triplicate. Controls (c7, c9, c11 and c12) from brain are labelled. The legend lists all samples left to right in the order they are presented in the graph. The brain readings of the IV low dosage (25, 26, 27), IP low dosage (45, 47, 48) and IV high dosage (34, 35, 36) are illustrated. Values represent the average of three readings for each treated animal or the average of all reading from control animals.

FIG. 11. As in FIG. 10, an ELISA plate was coated with a commercial rabies vaccine. Liver homogenates were diluted to various concentrations (ng/μl) and added to the ELISA plate (100 μl). Each treated and control sample was run in triplicate. Controls (c7, c9, c11 and c12) from the liver extract are indicated. The legend lists all samples left to right in the order they are presented in the graph. The results from high dosages (34, 36, 39) are illustrated. Values represent the average of three readings for each treated animal or the average of all reading from control animals.

FIG. 12. Mice were treated by IV injection of 1×10¹⁰ (1.E+10), 1.E+11, 1.E+12 and 1.E+13 vector genomes/kg body weight. Serum was measured for neutralizing antibodies to the rabies virus (IU/ml) by RFFIT. A standard rabies virus vaccine was used to vaccinate control mice. ⁺p<0.05 and ⁺⁺⁺p<0.0001 versus vaccine. *p<0.05, ***p<0.0001 versus 1.E+13 dose,

FIG. 13. Anti-rabies antibodies were detected in the brain with anti-human IgG:FITC (green) for the mice of the experiment of FIG. 12. Left panel, AAV-treated mouse; Right panel, untreated control mouse.

FIG. 14. Production of anti-rabies antibody in cat serum. Wild-type domestic cats were treated by IV injection of an AAV9 vector expressing the native CR57 human monoclonal antibody at one of two doses: High, 1×10¹³ vg/kg; Low, 2×10¹² vg/kg. Three weeks post-injection, serum was collected for detection of the human monoclonal antibody by ELISA using an anti-human secondary antibody. Untreated control cats expressed no CR57 antibody, while treated cats expressed the antibody in a dose-dependent manner.

### DETAILED DESCRIPTION

Disclosed are compositions, vectors, and methods for treating and preventing rabies lyssavirus infection in a subject in need thereof. The compositions, vectors, and methods can be further described based on the following definitions.

Unless otherwise specified or indicated by context, the terms “a,” “an,” and “the,” mean “one or more.” For example, “protein” or “domain” should be interpreted to mean “one or more proteins” and “one or more domains,” respectively.

As used herein, “about”, “approximately,” “substantially,” and “significantly” will be understood by persons of ordinary skill in the art and will vary to some extent depending on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “subject,” “host,” or “individual” typically refer to an animal at risk for acquiring an infection by rabies lyssavirus, such as human and non-human animals. The terms “subject,” “host,” or “individual” may be used interchangeably.

In certain embodiments are provided that the animal may be a human, companion animal, a domesticated animal, a feral animal, a food or feed-producing animal, a livestock animal, a game animal, a racing animal, a performance animal, or a sport animal. In particular the mammal is a human, bovine, e.g., cow, equine, e.g., horse, canine, e.g., dog, feline, e.g., cat, a caprine, e.g., goat, ovine, e.g., sheep, porcine, e.g., pig, other ungulate e.g., deer, or any other mammal.

As used herein the terms “antibody gene therapy” and “antibody molecular therapy” refer to the same procedure, product or process and are used interchangeably without any distinction or difference as embodied in this invention.

As used herein, the term, “rAAV gene therapy vector” refers to any pharmacological or biological agent used to induce a neutralizing antibody response to a pathogenic microorganism or the toxin or metabolite of a microorganism. As used herein the terms “rAAV Gene Therapy Vector” and “rAAV Molecular Therapy Vector” or any variants thereof are used interchangeably without any distinction or difference, as embodied in this invention.

The term “rAAV gene therapy vector” as used herein may be any adeno-associated virus, such as, but not limited to, a human adeno-associated virus, a bovine adeno-associated virus, a canine adeno-associated virus, a non-human primate adeno-associated virus, a chicken adeno-associated virus, or a porcine or swine adeno-associated virus, or any other adeno-associated virus derived vector, naturally occurring or synthetic may be used for molecular or gene therapy.

Rabies Lyssavirus

Rabies lyssavirus, formerly Rabies virus, is a neurotropic virus that causes rabies in humans and animals. Transmission can occur through contact with saliva of infected animals. Rabies lyssavirus has a cylindrical, bullet-shaped morphology. It is enveloped and has a single-stranded RNA genome with negative-sense. The RNA genome of the virus encodes five genes: nucleoprotein (N), phosphoprotein (P), matrix protein (M), glycoprotein (G) and the viral RNA polymerase (L). Upon viral entry into a host, the body produces virus neutralizing antibodies which bind and inactivate the virus. Specific regions of the G protein, which is present on the surface of the virus, have been shown to be most antigenic in leading to the production of virus neutralizing antibodies.

Polypeptides, Proteins, and Peptides

The disclosed vectors may be utilized to express polypeptides or proteins such as immunoglobulins against rabies lyssavirus. As used herein, polypeptide, proteins, and peptides comprise polymers of amino acids, otherwise referred to as “amino acid sequences.” A polypeptide or protein is typically of length ≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). However, the terms “polypeptide,” “protein,” and “peptide” may be used interchangeably herein.

The amino acid sequences contemplated herein may include one or more amino acid substitutions relative to a reference amino acid sequence (e.g., relative to any of SEQ ID NOs:1-119). In some cases, these substitutions may be conservative amino acid substitutions relative to the reference amino acid sequence. For example, a variant, mutant, or derivative polypeptide may include conservative amino acid substitutions and/or non-conservative amino acid substitutions relative to a reference polypeptide, which may include but is not limited to any of SEQ ID NOs:1-119. “Conservative amino acid substitutions” are those substitutions that are predicted to interfere least with the properties of the reference polypeptide. In other words, conservative amino acid substitutions substantially conserve the structure and the function of the reference protein. Table 1 provides a list of exemplary conservative amino acid substitutions.

TABLE 1

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. In contrast, non-conservative amino acid substitutions generally disrupt and/or alter (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in a reference amino acid sequence (e.g., any of SEQ ID NOs:1-119) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence that extends the half-life of the fusion polypeptide in serum. A “variant” of a reference polypeptide sequence may include a fusion polypeptide comprising the reference polypeptide.

A “fragment” is a portion of an amino acid sequence that is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise no more than 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise a range of contiguous amino acid residues of a reference polypeptide bounded by any of these values (e.g., 400-600 contiguous amino acid residues). Fragments may be preferentially selected from certain regions of a molecule. The term “at least a fragment” encompasses the full-length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of amino acid residue matches between at least two polypeptide sequences aligned using a standardized algorithm. Methods of polypeptide sequence alignment are well-known. Some alignment methods take into account conservative amino acid substitutions, non-conservative amino acid substitutions, deletions, and/or insertions. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” which is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence as defined by a particular SEQ ID number, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 contiguous amino acid residues of any reference sequence; or a fragment of no more than 15, 20, 30, 40, 50, 100, 150, 200, 250, 300, 350, 400, 450, 500, 550, 600, 650, at least 700, at least 750, at least 800, at least 850, at least 900, at least 950, or at least 1000 contiguous amino acid residues of any reference sequence; or over a range bounded by any of these values (e.g., a range of 500-600 amino acid residues). Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In other embodiments, a pair of polypeptides may show, for example, at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

Polynucleotides, Nucleic Acid, and Nucleic Acid Sequences

The disclosed vectors comprise polynucleotides for expressing polypeptides or proteins, such as immunoglobulins against rabies lyssavirus. The terms “polynucleotide,” “nucleic acid” and “nucleic acid sequence” refer to a polymer of DNA or RNA nucleotide of genomic or synthetic origin (which may be single-stranded or double-stranded and may represent the sense or the antisense strand). The polynucleotides contemplated herein may encode and may be utilized to express one or more immunoglobulins as disclosed herein.

The terms “nucleic acid” and “oligonucleotide,” as used herein, refer to polydeoxyribonucleotides (containing 2-deoxy-ribose), polyribonucleotides (containing ribose), and to any other type of polynucleotide that is an N glycoside of a purine or pyrimidine base. As used herein, the terms “A,” “T,” “C”, “G” and “U” refer to adenine, thymine, cytosine, guanine, uracil as a nucleotide base, respectively. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

A “fragment” of a polynucleotide is a portion of a polynucleotide sequence which is identical in sequence to but shorter in length than a reference sequence. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide; in other embodiments a fragment may comprise no more than 5, 10, 15, 20, 25, 30, 40, 50, 60, 70, 80, 90, 100, 150, 250, or 500 contiguous nucleotides of a reference polynucleotide; in further embodiments a fragment may comprise a range of contiguous nucleotides of a reference polynucleotide bounded by any of the foregoing values (e.g. a fragment comprising 20-50 contiguous nucleotides of a reference polynucleotide). Fragments may be preferentially selected from certain regions of a molecule. The term “at least a fragment” encompasses the full-length polynucleotide. A “variant,” “mutant,” or “derivative” of a reference polynucleotide sequence may include a fragment of the reference polynucleotide sequence.

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The term “promoter” as used herein refers to a cis-acting DNA sequence that directs RNA polymerase and other trans-acting transcription factors to initiate RNA transcription from the DNA or RNA (in an RNA virus) template that includes the cis-acting DNA of RNA sequence. A promoter may be “operably linked” to a coding sequence meaning that the promoter promotes transcription of the coding sequence, for example, as part of a vector.

Vectors

The disclosed subject matter relates to expression vectors and methods for using expression vectors. The term “vector” refers to some means by which DNA or RNA can be introduced into a host. There are various types of vectors including virus, plasmid, bacteriophages, cosmids, and bacteria. As used herein, a “viral vector” refers to recombinant viral nucleic acid that has been engineered to express a heterologous polypeptide (e.g., a recombinant immunoglobulin). The recombinant viral nucleic acid typically includes cis-acting elements for expression of the heterologous polypeptide. The recombinant viral nucleic acid typically is capable of being packaged into a virus that is capable of infecting a host cell. For example, the recombinant viral nucleic acid may include cis-acting elements for packaging. Preferably, the viral vector is not replication competent, is attenuated, or at least does not cause disease. The viral vector may naturally be non-pathogenic to the host. Alternatively, the viral vector may be genetically altered by modern molecular biological methods (e.g., restriction endonuclease and ligase treatment, and rendered less virulent than wild type), typically by deletion of specific genes. For example, the recombinant viral nucleic acid may lack a gene essential for production of infectious or virulent virus.

The recombinant viral nucleic acid may function as a vector for expressing an immunoglobulin against rabies lyssavirus by virtue of the recombinant viral nucleic acid containing foreign DNA or RNA. The recombinant viral nucleic acid, packaged in a virus, may be introduced into a vaccinee by standard methods for vaccination.

Numerous virus species can be used as the recombinant virus vectors for the composition disclosed herein. A preferred recombinant virus vector for a viral vaccine may include a recombinant adeno-associated virus vector (AAV) vector. Adeno associated virus (AAV) is a desirable vector for delivering therapeutic genes due to its safety profile and capability of long term gene expression in vivo.

A recombinant AAV vector (AAV viral particle) may comprise, packaged within an AAV capsid, a nucleic acid molecule containing a 5′ AAV inverted terminal repeat (ITR), the expression cassettes described herein and a 3′ AAV ITR. As described herein, an expression cassette may contain regulatory elements for an open reading frame(s) within each expression cassette and the nucleic acid molecule may optionally contain additional regulatory elements. Methods of generating AAV vectors have been described extensively in the literature and patent documents, including, e.g., WO 2003/042397; WO 2005/033321, WO 2006/110689; U.S. Pat. No. 7,588,772 B2. The source of AAV capsids may be selected from an AAV which targets a desired tissue. For example, suitable AAV may include, e.g., AAV9 (U.S. Pat. No. 7,906,111; US 2011-0236353-A1), rh10 (WO 2003/042397) and/or hu37 (see, e.g., U.S. Pat. No. 7,906,111; US 2011-0236353-A1). However, other AAV, including, e.g., AAV1, AAV2, AAV3, AAV4, AAVS, AAV6, AAV7, AAV8, (U.S. Pat. Nos. 7,790,449; 7,282,199) and others. AAV vectors for expressing immunoglobulins and targeting the CNS have been described. (See, e.g., US 20060034805A1, CN106470736A, EP2826860B1, U.S. Pat. No. 7,498,024B2, US20170080100A1, WO1995028493A1, AU2003231230A1 U.S. Pat. No. 8,034,331B2, EP2058401A1, U.S. Pat. No. 9,725,485B2, JP6387350B2, DE102014207498A1, U.S. Pat. No. 6,855,314B1, US20090069261A1 EP2212424B1, EP2029742B1, and EP2185712B1). However, other sources of AAV capsids and other viral elements may be selected, as may other immunoglobulin constructs and other vector elements.

Codon Optimization

The transgene expressed in the vectors disclosed herein (i.e., the immunoglobulin) may have the native polynucleotide sequence of an immunoglobulin or may have a polynucleotide sequence that has been modified. For example, the presently disclosed vectors may express polypeptides from polynucleotides that encode the polypeptides where the polynucleotides contain codons that are optimized for expression in a particular host. For example, presently disclosed vectors may include one or more polypeptides where the encoding polynucleotide sequence is optimized to include codons that are most prevalent in a human or non-human animal.

Rabies Immunoglobulins

The vectors disclosed herein can be utilized to express rabies immunoglobulins in a subject in need thereof. The term “immunoglobulin” is used herein to include antibodies, and functional fragments thereof. Antibodies may exist in a variety of forms including, but not limited to, monoclonal antibodies, camelid single domain antibodies, intracellular antibodies, recombinant antibodies, multispecific antibodies (e.g., bispecific antibodies), and antibody fragments, such as, Fv, Fab, F(ab)₂, F(ab)₃, Fab′, F(ab′)₂, single chain variable fragment antibodies (scFv), tandem/bis-scFv, Fc, pFc′, scFvFc (or scFv-Fc), disulfide Fv (dsfv), and bispecific antibodies (bc-scFv) such as BiTE antibodies. The term “antibody fragment” refers to at least a portion of the variable region of the immunoglobulin that binds to its target, e.g., an epitope of rabies lyssavirus. Immunoglobulins against rabies lyssavirus are known in the art. (See, e.g., U.S. Pat. Nos. 7,579,446; 6,890,532; German (DE) Published Application No. 4006630A1; the contents of which are incorporated herein by reference in their entireties).

Antigens and Dose

The disclosed methods typically include administering an AAV vector that expresses an anti-rabies immunoglobulin. In some embodiments, the disclosed methods may include an additional step of administering a rabies lyssavirus antigen, for example to induce an immune response against the antigen.

As such, the compositions disclosed herein optionally may include an antigen or a plurality of antigens against rabies lyssavirus. A “plurality” or antigens as used herein means “more than one” and may mean more than 1, 2, 3, 4, 5, 10, 25, 50, or 100 antigens.

As such, the composition, vectors, and methods disclosed herein may utilize a protein, polypeptide, peptide, or plurality thereof as an antigen. The compositions, vectors, and methods may be utilized to induce an antibody response and/or a cell-mediated response against infection by rabies lyssavirus.

Suitable antigens may include polypeptides, peptides, or panels thereof that comprise one or more epitopes of a protein expressed by a pathogen associated with a disease. For example, suitable polypeptides, peptides, or panels thereof may comprise one or more epitopes of a protein associated with a pathogen. Suitable polypeptides may comprise the full-length amino acid sequence of a corresponding protein of a pathogen or a fragment thereof. For example, suitable fragments may include 5-2000 amino acids (or from 5-1000, 5-100, 5-50, 5-25, 5-15, 10-2000, 10-1000, 10-50, 10-25, or 10-15 amino acids) and include at least one epitope of the protein from which the fragment is derived. Suitable antigens for the compositions, vectors, and methods may include or express a plurality of peptides derived from a protein of a pathogen. For example, a suitable antigen may comprise a plurality of at least 2, 3, 4, 5, 10, 25, 50, 100, or more different peptides comprising at least about a 10-20 amino acid sequence from a protein of a pathogen. The different peptide antigens may overlap at the N-terminus, the C-terminus, or both termini with at least one other peptide antigen of the composition, for example, by at least about 1, 2, 3, 4, 5, 6, 7, 8, 9, or 10 amino acids.

Prime-Boost Regimen

As used herein, a “prime-boost regimen” refers to a regimen in which a subject is administered a first composition and then after a determined period of time (e.g., after about 2, 3, 4, 5, or 6 weeks), the subject is administered a second composition, which may be the same or different than the first composition. The first composition (and the second composition) may be administered one or more times. The disclosed methods may include priming a subject with a first composition by administering the first composition at least one time, allowing a predetermined length of time to pass (e.g., at least about 2, 3, 4, 5, or 6 weeks), and then boosting by administering the same composition or a second, different composition.

For example, the methods may include administering a first pharmaceutical composition and optionally may include administering a second pharmaceutical composition to augment or boost an immunogenic response induced by the first pharmaceutical composition. The first and second pharmaceutical compositions may be the same or different. The optionally administered second pharmaceutical composition may be administered prior to, concurrently with, or after administering the first pharmaceutical composition. In some embodiments, the first composition is administered and then the second composition is administered after waiting at least about 2, 3, 4, 5, or 6 weeks. The first composition (and the second composition) may be administered one or more times.

Characterization of a Response in Subjects

The pharmaceutical compositions disclosed herein may be delivered to subjects at risk for acquiring an infection by rabies lyssavirus. In order to assess the efficacy of an administered composition or vaccine, the immune response can be assessed by measuring the production of antibodies to particular epitopes of rabies lyssavirus and/or cell-mediated responses against rabies lyssavirus. Antibody responses may be measured by assays known in the art such as ELISA. Immune responses also may be characterized by physiological responses. (See Li et al., Vaccine 28 (2010) 1598-1605; and Stemke-Hale et al., Vaccine 2005 Apr. 27; 23(23):3016-25, the content of which are incorporated herein by reference in their entireties.) Immune response also may be measured by reduction in pathological responses after challenge with rabies lyssavirus, or reduction in titer or load as measured using methods in the art including methods that detect nucleic acid of the pathogen.

As used herein, an “immune response” may include an antibody response (i.e., a humoral response), where an individual produces antibodies against rabies lyssavirus (e.g., IgG (IgY), IgA, IgM, or other antibody isotypes). As used herein, an “immune response” also may include a cell-mediated response, for example, a cytotoxic T-cell response against cells expressing foreign peptides derived from an administered antigen in the context of a major histocompatibility complex (MHC) class I molecule.

As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”).

As used herein, “viral load” is the amount of virus present in a sample from a subject infected with the virus. Viral load is also referred to as viral titer or viremia. Viral load can be measured in variety of standard ways including copy Equivalents of the viral RNA (vRNA) genome per milliliter individual sample (vRNA copy Eq/ml). This quantity may be determined by standard methods that include RT-PCR.

Pharmaceutical Compositions

The compositions disclosed herein may include pharmaceutical compositions comprising the presently disclosed recombinant proteins and/or vectors for expressing the presently disclosed recombinant proteins, which are formulated for administration to a subject in need thereof. Such compositions can be formulated and/or administered in dosages and by techniques well known to those skilled in the medical arts taking into consideration such factors as the age of the particular subjects and the route of administration.

The disclosed compositions may include liquid formulations, solid formulations, lyophilized forms, or suspensions. In some embodiments, the formulation may further comprise a delivery device.

The disclosed compositions may include additional components such as carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The disclosed compositions may be administered in an amount sufficient to induce an immune response for protecting against infection. Inducing a protective response may include inducing sterilizing immunity against a pathogen (e.g., against rabies lyssavirus), or reducing the effects of the pathogen.

The compositions disclosed herein may be delivered via a variety of routes. Typical delivery routes include parenteral administration (e.g., intradermal, intramuscular, intraperitoneal, or subcutaneous delivery), intranasal, intravenous, oral, and ocular (via eyedrop). Formulations of the pharmaceutical compositions may include liquids (e.g., solutions and emulsions), sprays, and aerosols.

The compositions disclosed herein may be co-administered or sequentially administered with other immunological, antigenic or vaccine or therapeutic compositions, including an adjuvant, or a chemical or biological agent given in combination with an antigen to enhance immunogenicity of the antigen.

Adjuvants. The compositions disclosed herein optionally include an adjuvant. The term “adjuvant” refers to a compound or mixture that enhances an immune response. An adjuvant can serve as a tissue depot that slowly releases the antigen and also as a lymphoid system activator that non-specifically enhances the immune response. Examples of adjuvants that may be utilized in the disclosed compositions include but are not limited to, co-polymer adjuvants (e.g., Pluronic L121® brand poloxamer 401, CRL1005, or a low molecular weight co-polymer adjuvant such as Polygen® adjuvant), poly (I:C), R-848 (a Th1-like adjuvant), resiquimod, imiquimod, PAM3CYS, aluminum phosphates (e.g., AlPO₄), loxoribine, potentially useful human adjuvants such as BCG (Bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin (e.g., Quil-A), mineral gels such as aluminum hydroxide, surface active substances such as lysolecithin, pluronic polyols, polyanions, peptides, oil or hydrocarbon emulsions in water (e.g., MF59 available from Novartis Vaccines, Montanide ISA 720, or Montanide ISA 71 VG), keyhole limpet hemocyanins, and dinitrophenol.

Antibody Gene Therapy

In some embodiments, the disclosed compositions, rAAV vectors, and methods may be utilized for inducing protection or therapy against a pathogenic infection, for example, by rabies lyssavirus, via antibody gene therapy. The compositions of rAAV may be formulated for administration to a human or any other animal accordingly. With respect to dosages, routes of administration, formulations, and uses for recombinant virus vectors and expression products therefrom, the disclosed composition may be used for parenteral or mucosal administration, preferably by intravenous, intraperitoneal, intradermal, subcutaneous or intramuscular routes. When mucosal administration is used, it is possible to use oral, ocular or nasal routes.

The formulations comprising the rAAV antibody molecular therapy vector of interest, can be prepared in accordance with standard techniques well known to those skilled in the pharmaceutical or biopharmaceutical arts. Such formulations can be administered in dosages and by techniques well known to those skilled in the medical arts taking into consideration such factors as the age, sex, weight of an animal and the route of administration. The formulations can be administered alone or can be co-administered or sequentially administered with compositions.

The formulations may be present in a preparation for parenteral, intravenous, intraperitoneal, subcutaneous, intradermal, intramuscular (e.g., injectable administration) such as sterile suspensions or emulsions. In such formulations the rAAV vector may be in admixture with a suitable carrier, diluent, or excipient such as sterile water, physiological saline, or the like. The formulations can also be lyophilized or frozen. The formulations can contain auxiliary substances such as wetting or emulsifying agents, pH buffering agents, adjuvants, preservatives, and the like, depending upon the route of administration and the preparation desired.

Any active component may be admixed with rAAV vectors under sterile conditions with a physiologically acceptable carrier and any preservative, buffers, propellants, or absorption enhancers as may be needed.

An immunological effective amount, as used herein refers to an amount or concentration of the rAAV gene therapy vector encoding and expressing the recombinant protein of interest, that when administered to a subject, produces an immune response to the pathogen or its toxin or metabolite. The rAAV molecular therapy vector of the present disclosure may be administered to a human or other animal either alone or as part of an immunological composition.

In some embodiments of the disclosed methods of rAAV gene therapy, the vectors may be administered at a dosage from about 10⁴ to about 10¹⁶ vg/kg body weight. In one aspect the dose administered to the animal is about, or at least about, 10⁴ vg/kg. In another aspect the dose administered to the animal is about, or at least about, 10⁵ vg/kg. In yet another aspect, the dose of administered to the animal is about, or at least about, 10⁶ vg/kg. In another aspect the dose of Ad-vector administered to the animal is about, or at least about, 10⁷ vg/kg. In another aspect the dose administered to the animal is about, or at least about, 10⁸ vg/kg. In yet another aspect, the dose administered to the animal is about, or at least about, 10⁹ vg/kg. In yet another aspect, the dose administered to the animal is about, or at least about, 10¹⁰ vg/kg. In yet another aspect, the dose administered to the animal is about, or at least about, 10¹¹ vg/kg. In yet another aspect, the dose administered to the animal is about, or at least about, 10¹² vg/kg. In yet another aspect, the dose administered to the animal is about, or at least about, 10¹³ vg/kg. In yet another aspect, the dose administered to the animal is about, or at least about, 10¹⁴ vg/kg. In yet another aspect, the dose administered to the animal is about, or at least about, 10¹⁵ vg/kg. In yet another aspect, the dose administered to the animal is about, or at least about, 10¹⁶ vg/kg. Furthermore, an embodiment of this disclosure instructs the use of such AAV-antibody gene doses. One of skill in the art understands that an effective dose in a mouse may be scaled for larger animals such as dogs, horses, pigs, etc. In that way, through allometric scaling (also referred to as biological scaling) a dose in a larger animal may be extrapolated to obtain an equivalent dose based on body weight or body surface area of the animal.

Recombinant Compositions, Vectors, and Methods for Treating and Preventing Rabies Lyssavirus Infection

Disclosed are compositions, vectors, and methods for treating and preventing rabies lyssavirus infection in a subject in need thereof, including rabies lyssavirus encephalitis. The disclosed compositions relate to anti-rabies immunoglobulins and vectors for expressing anti-rabies immunoglobulins such as adeno-associated virus (AAV) vectors that express anti-rabies immunoglobulins in the subject. In some embodiments, the AAV vectors express anti-rabies immunoglobulins systemically in the subject. Preferably, the AAV vectors express anti-rabies immunoglobulins in the nervous system of the subject, such as the central nervous system (CNS) and/or the peripheral nervous system. In some embodiments, the disclosed methods relate to treating and/or preventing an infection by rabies lyssavirus in a subject in need thereof, the methods comprising administering to the subject a dose of an adeno-associated virus (AAV) vector that expresses an immunoglobulin that binds and neutralizes rabies lyssavirus in the subject.

The disclosed compositions and vectors may be administered to a subject in need thereof by any suitable method. In embodiments are provided methods for treatment of an animal wherein the rAAV gene therapy vector is administered by intravenous, intraperitoneal, intradermal, subcutaneous, intramuscular, oral, topical, intranasal, intravaginal, rectal, intrathecal, mucosal, inhalation or intracerebral routes and antibodies to a pathogen are produced by cells of organs including: liver, kidney and organs of the urogenital system, muscle, bone, bone marrow and the hematopoietic system, organs of the immune system, organs of the reproductive system, lung, skin, eyes, blood, blood cells, endocrine organs or the gastrointestinal system where antibody genes express antibodies and/or antibody fragments that bind and neutralize rabies lyssavirus and abrogate its pathogenic effects by prevention or treatment of infection.

In some embodiments, the disclosed compositions and vectors are administered to the subject via injection. Suitable routes of administration may include, but are not limited to, intravenous, intramuscular, subcutaneous, oral, or nasal.

The disclosed compositions and vectors relate to anti-rabies immunoglobulins and expression thereof. In some embodiments, the anti-rabies immunoglobulins bind to Glycoprotein G of rabies lyssavirus and preferably neutralize the virus.

The disclosed composition and vectors relate to anti-rabies immunoglobulins and expression thereof. Suitable immunoglobulins may include, but are not limited to monoclonal antibodies, camelid antibodies, single domain antibodies (sdAb), intracellular antibodies, recombinant antibodies, multispecific antibodies, Fv, Fab, F(ab)₂, F(ab)₃, Fab′, Fab′-SH, F(ab′)₂, single chain variable fragment antibodies (scFv), di-scFv, Fc, pFc′, and scFvFc. In some embodiments, the immunoglobulin is a human antibody or a fragment thereof.

In the disclosed methods for treating and/or preventing an infection by rabies lyssavirus in a subject in need thereof, the subject is administered a dose of an AAV vector in order express a suitable amount of an anti-rabies antibody in the subject. In some embodiments of the disclosed methods, the AAV vector is administered to the subject at a dose of no more than about 10¹⁴, 10¹³, 10¹², 10¹¹, 10¹⁰, 10⁹, 10⁸, 10⁷, 10⁶, 10⁵, and 10⁴ viral genomes (vg)/kg body weight of the subject, or within a dose range bounded by any of these values (e.g., 10¹⁴-10¹⁰ vg/kg, or 10⁸-10⁴ vg/kg, or 10⁶-10⁴ vg/kg). Preferably, after the AAV vector is administered to the subject, the subject expresses an effective amount of an anti-rabies immunoglobulin for treating and preventing infection by rabies lyssavirus. In some embodiments, the subject is administered a dose of the AAV vector which is effective for expressing the immunoglobulin in the subject at a titer of at least about 1:100, 1:500, 1:10000, 1:50000, or 1:100000, (or at a concentration of at least about 5 IU/ml, 10 IU/ml, 50 IU/ml, 100 IU/ml, 500 IU/ml, or 1000 IU/ml).

In some embodiments, the disclosed methods for treating and preventing infection by rabies lyssavirus in a subject may be advantageous over other methods known in the art.

The disclosed methods may provide rapid protection against rabies lyssavirus after treatment whereby neutralizing antibody titers in a subject that is subjected to the disclosed methods may be at least about 1:5, 1:10, 1:50, 1:100, 1:200, 1:500, 1:1000, 1:2000, 1:5000, or 1:10000 (or 0.5 IU/ml, 1 IU/ml, 5 IU/ml, 10 IU/ml, 50 IU/ml 100 IU/ml, 500 IU/ml, or 1000 IU/ml) as soon as 1, 2, 4, 6, 8, 10, 12, 14, 16, 18, 20, 22, 24, 36, 48, 60, 72, 96, 120, 144, 168, 192, 216, 240, or 256 hours after treatment. This is important because in post-exposure therapy for rabies, if the bite wound is in the face the time between exposure and fatal encephalitis can be days. Traditional immunization may not provide adequate protection before infection is fatal. For treatment of patients with known encephalitis, the disclosed treatment method may provide immediate neutralization of virus in brain and may prevent the massive infection of brain cells and destruction of so much of the CNS that the patient dies.

The disclosed methods may provide rapid protection against rabies lyssavirus after a single treatment. The disclosed methods may require only a single treatment to achieve protective levels of neutralizing antibody in a subject, whereas other methods may require multiple treatments over a period of weeks to months, during which time subjects are not protected. This plus early protection relieves the patient and medical team from frequent and constant attention, anxiety, and cost.

The disclosed methods may provide life time protection against rabies lyssavirus. The disclosed methods may provide for protection against rabies lyssavirus for many years, potentially for the life of the subject. Other methods in the art provide protection against rabies lyssavirus for limited times ranging from 1 to 3 years. The lapse of protection during these periods for any one subject is not known. If a subject who is immunized with time limited vaccines is exposed, the standard is to determine the protective titer at that time, adding to concern and expense. The lack of protective titer requires boosting adding to anxiety, medical care and expense. The disclosed methods may eliminate all of this added treatment and expense.

The disclosed methods may be utilized to induce maternal antibodies and protect children. When a mother is immunized her progeny may/or not be protected by maternal antibodies delivered prenatally (human) or in the colostrum (other mammals). The period of this passive protection varies widely and the exact interval between protection and loss of the passive immunity is not known. Therefore, immunization of these individuals with a live virus vaccine cannot be protective during the period of passive immunity (virus neutralization) and after passive immunity is no longer protective the individual is fully susceptible to infection. Even if live virus is given after the passive immunity is gone, it requires 2-4 weeks for the young to be protected. The disclosed methods may be performed during the time when passive immunity is active and the disclosed methods still will provide indefinite protection eliminating the need for guessing, tittering and reimmunizations.

The disclosed methods may be utilized to induce antibodies in the immunocompromised. Subjects without natural or induced capacity to mount a protective immune response to traditional vaccination are not protected, and may not be aware of their lack of protection. The disclosed methods may be performed to induce antibody production without requiring that a subject mount a protective immune response, as in traditional, antigen-based immunization.

### ILLUSTRATIVE EMBODIMENTS

The following embodiments are illustrative and should not be interpreted to limit the scope of the claimed subject matter.

Embodiment 1. Compositions and methods comprising and/or utilizing recombinant adeno-associated virus (rAAV) vectors that cross the blood-brain-barrier and transfect cells of the nervous system and other organs and express inserted genes encoding entire antibodies or antibody fragments that are capable of binding rabies lyssavirus and neutralizing its pathogenic effects that cause diseases of the nervous system, directly or indirectly, as well as other organs in animals and humans. AAV vector capsids that are superior for CNS gene transfer may be used to achieve high levels and widespread production of anti-pathogen antibodies in the CNS and other organs protected by blood-barriers.

Embodiment 2. The compositions and methods of embodiment 1, wherein the rAAVs are designed to selectively pass through the intact blood-brain-barrier (BBB) so that the transgene encoding genes for antibodies and/or antibody fragments can be expressed directly in the central nervous system (CNS) and/or the peripheral nervous system (PNS), so that such antibodies and/or antibody fragments can bind and neutralize rabies lyssavirus in situ, thereby preventing and/or treating and/or preventing rabies lyssavirus infection. Such AAV vectors used in the disclosed methods are not limited to any one particular type of AAV that may be used to achieve the practice of AAV transduction of the CNS and PNS for antibody expression and pathogen neutralization.

Embodiment 3. The compositions and methods of embodiment 1 or 2, wherein the vectors express two or more antibodies. Furthermore, AAV vectors used herein may express one or more antibody genes that produce one or more different antibodies that are needed to bind and neutralize any number of genetic variants of pathogens. Such variants may exist in various regions such that multiple antigenic epitopes require more than one antibody to bind and neutralize these variants or new genetic variants that may exist. This invention may neutralize such variants or multiple variants by expressing one or more genes expressing varying antibodies that bind and neutralize such genetic variants of any pathogen.

Embodiment 4. The compositions or methods of any of the foregoing embodiments, wherein the rAAV vector includes combinations of promoters and enhancers and/or other elements that improve antibody gene expression so as to provide the optimal adeno-associated virus (rAAV)-based gene therapy delivery vector constructs to express antibody transgenes that express antibodies or antibody fragments that neutralize rabies lyssavirus and treat or prevent infection

Embodiment 5. The compositions or methods of any of the foregoing embodiments, wherein the rAAV is designed to not only pass through the intact blood-brain-barrier and transfect and express antibodies in the nervous system, but also transfect and express antibody and antibody fragment genes in organs other than the nervous system so that the transgene encoding genes for antibodies and/or antibody fragments can be expressed in both the nervous system and visceral organs. Thus, antibody gene therapy so designed and described in this invention is capable of neutralizing any pathogens that may infect organs outside of the CNS and PNS so as to prevent such pathogens from invading the CNS or PNS, as well as treat or prevent infection that advance to or otherwise affect the CNS or PNS of animals or humans.

Embodiment 6. The compositions or methods of any of the foregoing embodiments, wherein whole antibodies, antibody fragments of all types, whether naturally occurring or synthetic, that bind and neutralize rabies lyssavirus, for prevention and/or treatment of infectious diseases of humans and animals, particularly prevention and/or treatment of neuropathogenic infections such as, but not limited to rabies disease. All of these forms of antigen binding antibodies can be used in the present invention.

Embodiment 7. The compositions or methods of any of the foregoing embodiments, wherein the rAAV vector expresses one or more antibody genes that interfere with the growth, spread within a host or shedding and transmission between hosts of rabies lyssavirus.

Embodiment 8. The compositions or methods of any of the foregoing embodiments, wherein the rAAV is designed to not only pass through the intact blood-brain-barrier, but also the blood barriers that prevent antibodies from entering the visual system or reproductive and other organs, so that antibodies and/or antibody fragments generated by antibody gene therapy can bind and neutralize pathogenic microorganisms that infect all of these blood-barrier protected organs thereby preventing and or treating such diseases of humans and animals that are not effectively treated by traditional vaccines.

Embodiment 9. The compositions or methods of any of the foregoing embodiments, wherein the rAAV vector inserts its antibody expressing genome into host cells extra-chromosomally in CNS and/or PNS cells and cells outside of the CNS and/or PNS, by which it expresses the antibody gene for long periods, even for as long as the life of humans and animals, thus eliminating the need for secondary immune stimulation, commonly referred to as booster immunization and repeated re-dosing to maintain protective antibody titers as is required of traditional vaccines.

Embodiment 10. The compositions or methods of any of the foregoing embodiments, wherein the rAAV vectors express inserted genes encoding entire antibodies or antibody fragments that are capable of binding various weak foreign antigens, particularly weak antigens of neuropathogens, for which traditional vaccines are deficient or ineffective in inducing protective antibody responses and these antibodies are able to bypass the blood-barriers and protect the nervous, visual and reproductive and other organs and prevent or treat infections of these organs in animals and humans.

Embodiment 11. The compositions or methods of any of the foregoing embodiments, wherein the rAAV vectors express inserted genes encoding entire antibodies or antibody fragments that are capable of binding and blocking various deleterious endogenous “self” antigens, especially “self” antigens produced by bio-mimicry from pathogens which traditional vaccines are deficient or ineffective in inducing protective antibody responses and these antibodies are able to bypass the blood-barriers and protect the nervous, visual, reproductive and other organs of animals and humans from such infections.

Embodiment 12. The compositions or methods of any of the foregoing embodiments, wherein the rAAV vector selectively transfects the CNS, PNS, liver, kidney and organs of the urogenital system, muscle, bone, bone marrow and the hematopoietic system, organs of the immune system, organs of the reproductive system, lung, skin, eyes, blood, blood cells, endocrine organs, gastrointestinal and other organ systems where antibody gene therapy genes as described in this invention express antibodies and/or antibody fragments that bind and neutralize pathogens and abrogate their pathogenic effects thereby prevent and/or treat of diseases of these organs and prevent or treat direct or indirect diseases of the nervous system.

Embodiment 16. The compositions or methods of any of the foregoing embodiments, wherein the rAAV vector transfect various organs, including, but not limited to the CNS and/or PNS and express inserted genes encoding entire antibodies or antibody fragments that are capable of binding various microorganisms and neutralizing their pathogenic effects and/or their toxins that cause neuropathic diseases in mammals including: humans (Homo sapiens) and other members of the Order Primates, Canidae, Felidae, Suidae, Bovidae, Equidae, Ovadie, Camalids and other members of the Class Mammalia, and all avian species.

### EXAMPLES

The following examples are illustrative and should not be interpreted to limit the claimed subject matter.

### Example 1—Construction of a Recombinant Adeno-Associated Virus Expressing an Anti-Rabies Lyssavirus Antibody

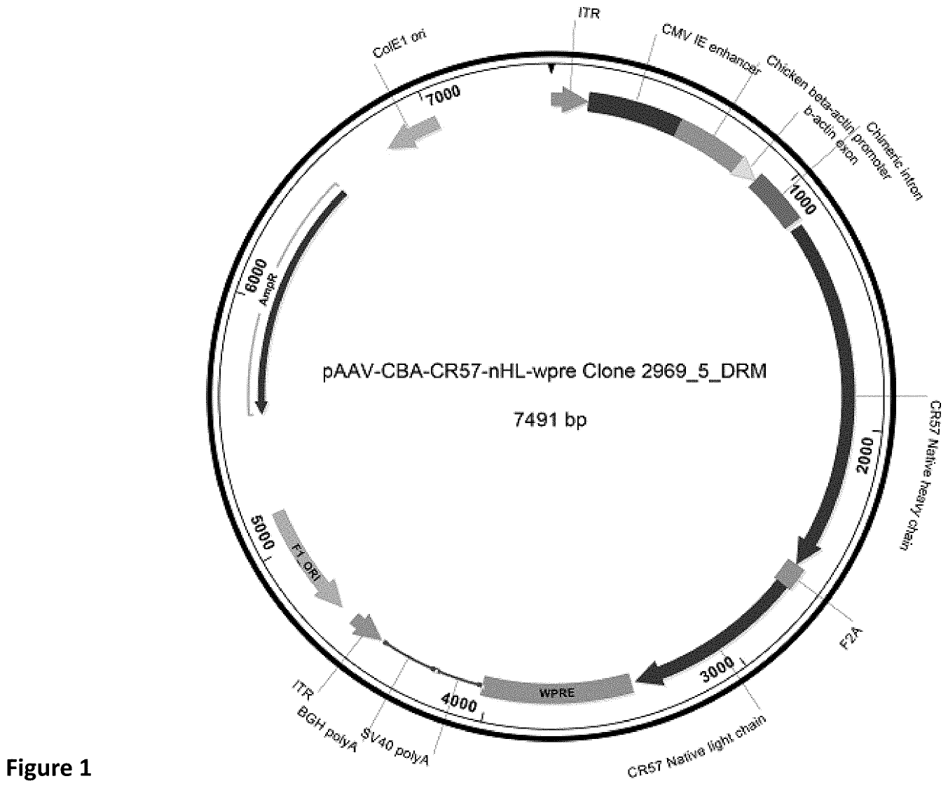

Recombinant adeno-associated virus (AAV) vectors were used to express anti-rabies neutralizing antibodies or antibody fragments. (See FIG. 1). As an exemplary AAV vector, the inventors utilized AAV9. However, other AAV vectors may similarly be utilized to express anti-rabies neutralizing antibodies or antibody fragments. Other suitable AAV vectors may include, but are not limited to AAV-AS42 and AAV-HP.B43. These and other variants of AAV may be used in the disclosed treatment and prevention methods. AAV expression sequence may be constructed with any number of promoters, enhancers and/or other elements to enhance expression of a protein product of a gene of interest. For example, a rAAV may incorporate the promoter chicken B-actin promoter fused to the cytomegalovirus enhancer and/or, carrying the rabbit beta-globin polyadenylation signal. Different designs of these AAV vectors and others may be more potent and/or efficient in expressing antibody genes. AAV vectors can be tested in cell culture at different multiplicities of infection (MOI). The AAV vector that yields the highest antibody production may be used in animal trials. Vector titers can be determined by quantitative PCR (qPCR) of vector genomes using specific primers (with or without probes).

### Example 2—Use of a Recombinant Adeno-Associated Virus Expressing an Antibody Against Rabies Lyssavirus to Prevent Rabies Prophylactically and Treat Patients Exposed to Rabies

AAV Vector Expressing Anti-rabies Antibody Genes: The inventors found that adeno-associated virus vectors can be utilized to carry antibody genes and express those genes to produce rabies neutralizing antibodies in the central nervous system (CNS) after systemic delivery. The rAAV vectors encode the rabies antibody heavy and light chains fused by a 2A peptide that mediates ribosomal skipping to produce both proteins from a single strong promoter (FIG. 1). The transgene size of 2,173 bases can be easily accommodated in the context of a single stranded AAV vector carrying a promoter and a polyadenylation signal.

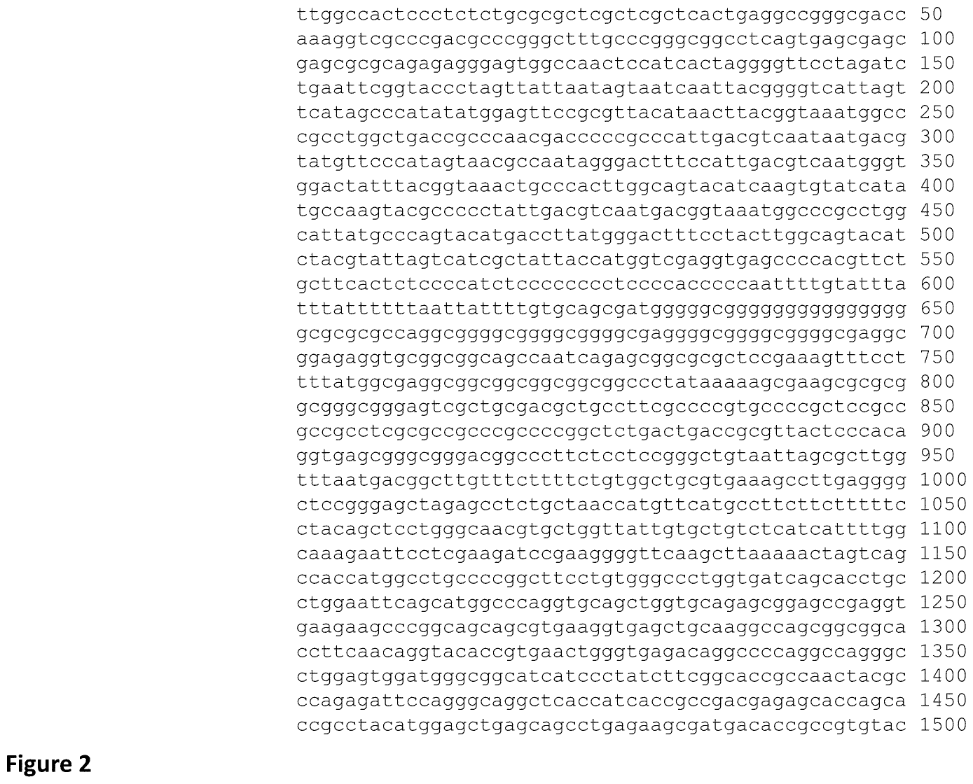

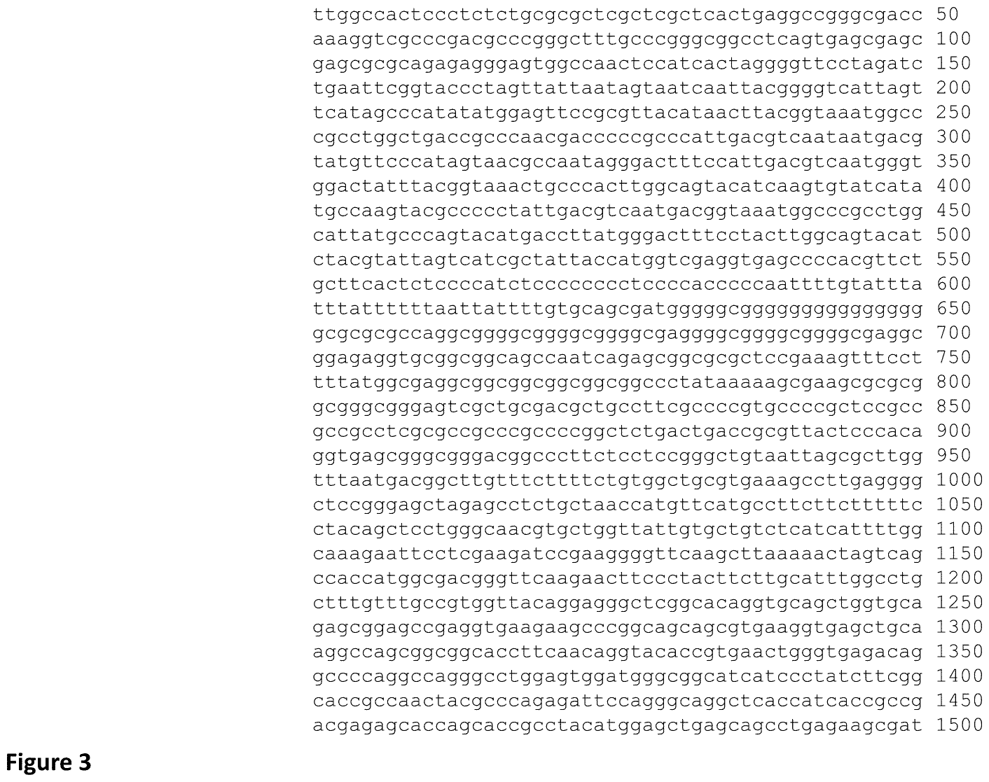

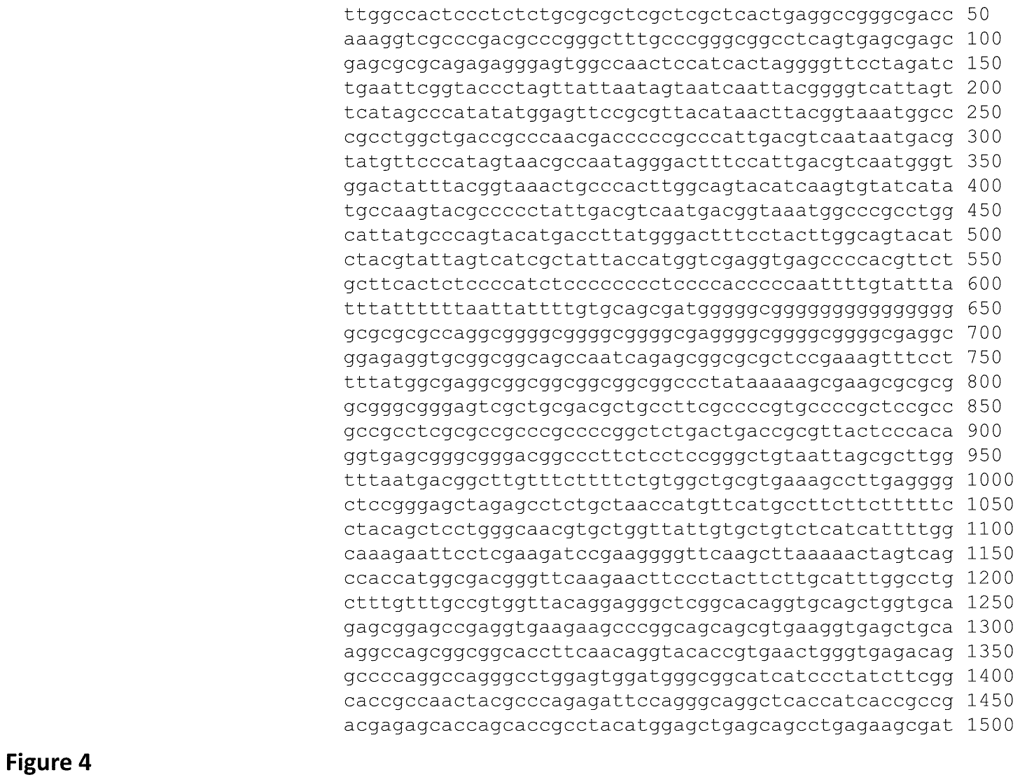

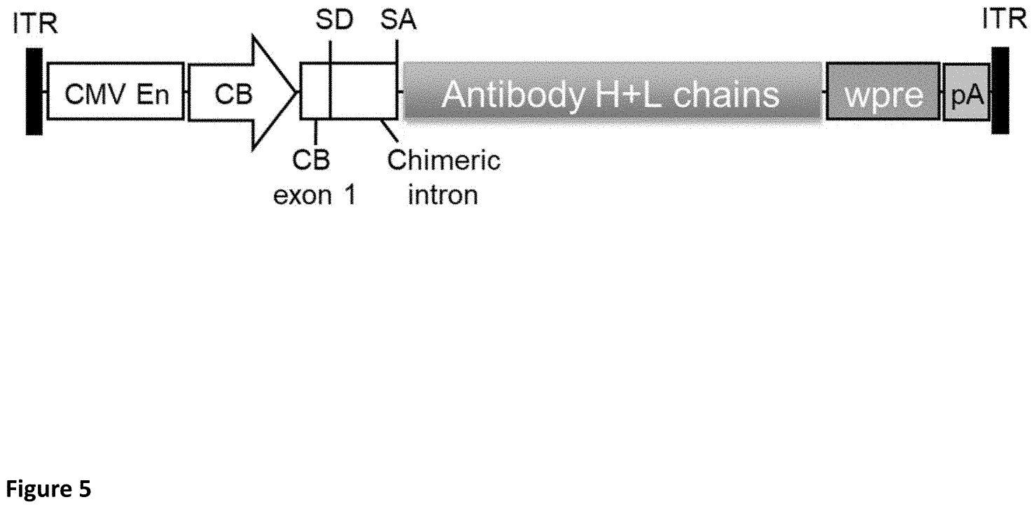

Anti-rabies antibody gene and expression cassette: As illustrated in FIGS. 2-4, the inventors inserted the following gene sequences into an AAV vector: SEQ pAAV-CBA-CR57-nHL-wpre Clone 2969_5_DRM, SEQ pAAV-CBA-CR57v1.1-wpre Clone 2970_2, and SEQ pAAV-CBA-CR57v3.1-wpre Clone 2971. The expression cassette is illustrated in FIGS. 1 and 5. These anti-rabies antibody genes are exemplary and other anti-rabies antibody genes may be utilized in the disclosed methods and composition. It should be understood that although the present example described the use of a single gene that expresses one anti-rabies antibody, it is also within the scope of the disclosed methods and compositions to express multiple genes that encode multiple antibodies that might be needed to bind and neutralize multiple genetic variants of a given rabies virus pathogen. It should be understood that genes that express anti-rabies antibodies can be of many cDNA sequences depending on the rabies virus mutants that are being treated or prevented. Furthermore, any number of combinations of these antibody gene sequences may be used to generate the most effective neutralizing antibodies, as discussed in Bakker, et al J. Virology 79, (2005) 9062-9068. AAV vectors expresses anti-rabies antibodies that protect from rabies infection or that can be used to express anti-rabies antibodies in the nervous system of patients exposed to rabies.

Adeno-associated virus Vectored Vaccine Design, Construction and Production: Recombinant AAV-RAB vector was used to encode rabies neutralizing antibody and demonstrate the production of anti-rabies antibody in vitro and in vivo. The inventors investigated different designs of AAV-RAB to examine the effect of different signal peptides. The designs that were tested included SEQ pAAV-CBA-CR57-nHL-wpre Clone 2969_5_DRM, SEQ pAAV-CBA-CR57v1.1-wpre Clone 2970_2, and SEQ pAAV-CBA-CR57v3.1-wpre Clone 2971. These designs were tested in cell culture at different multiplicities of infection (MOI). The design that yield the highest antibody production was used to produce AAV vectors for experimental animal trials. Gene sequences may include species specific constant regions of heavy and light chains of an antibody to eliminate immune reaction to antibodies recognized as not “self”, thus limiting the duration of effectiveness.

Antibody Expression in Cell Cultures: Antibody processing and expression levels were tested in HEK293 cells after transfection with plasmids containing different designs of the AAV vector backbone. The AAV-RAB vector constructs were tested in human HEK-293 cells grown in Minimum Essential Medium with 10% fetal calf serum at 37° C. in 5% CO₂. Cells were plated to a density of 3×10⁵ cells/well in 12-well dishes and transfected using a standard transfection method. Growth medium was harvested at 3 days post-transfection. Expression levels in media were assessed by ELISA using recombinant rabies G protein as capture antigen and live rabies virus neutralization conducted at the Centers for Disease Control and Prevention (CDC).

Antibody Assays: Anti-rabies antibody expression was tested in treated HEK-293 cell cultures to determine antibody neutralization. Cell culture supernatant was assayed for anti-rabies antibodies using the Rapid Fluorescent Focus Inhibition Test (RFFIT). Briefly, virus was incubated with culture supernatants at different dilutions and then added to cells. After 20 h, cells were stained with anti-rabies FITC antibodies to observe infection. Typically, 20 fields are observed on an 8-well slide. If all 20 fields had fluorescent staining suggesting infection with rabies virus, then there is no neutralization. If all 20 fields are devoid of fluorescence, then it corresponds to complete neutralization. Intermediate fields of infection between all or none are so noted.

Anti-Rabies Neutralizing Antibodies Supernatants from HEK-293 Cells Transfected with Plasmids Expressing Rabies Antibody Genes. Three constructs (CR57-1 (SEQ 2970), CR57-3 (SEQ 2971) and CR57 Native (SEQ 2969)) were transfected into HEK-293 cells and compared with untreated cells. CR57 comprises the native leader sequence of the CR57 monoclonal anti-rabies antibody while CR57-1 and CR57-3 comprise heterologous leader sequences.

TABLE 1
Anti-Rabies Neutralizing Antibodies Supernatants from HEK-293 Cells
Transfected with Plasmids Expressing Rabies Antibody Genes

| Repli- | | *Number of positive fields (of 20) | | | | Supernatant |
|---|---|---|---|---|---|---|
| cate | ID | None | 1:5 | 1:25 | 1:125 | dilution |
| 1 | CR57-1 | 7 | 20 | 20 | 20 | |
| | CR57-3 | 20 | 20 | 20 | 20 | |
| | CR57-native | 0 | 1 | 20 | 20 | |
| | untreated | 20 | 20 | 20 | 20 | |
| 2 | CR57-1 | 0 | 0 | 10 | 20 | |
| | CR57-3 | 15 | 20 | 20 | 20 | |
| | CR57-native | 0 | 0 | 5 | 20 | |
| | untreated | 20 | 20 | 20 | 20 | |

The results in Table 1 demonstrate neutralization activity in cells transfected with CR57-Native and CR-57-1 constructs. Cell culture supernatants were tested undiluted or diluted as indicated. The number of microscopic fields that show active rabies infected cells were recorded and scored as shown to indicate the rabies virus neutralization as shown in this table. The CR57-Native showed the most effective rabies neutralization and this construct was used in the mouse trials.

### Example 3. Responses of Mice to an Adeno-Associated Virus Vector Expressing the Antibody to Rabies Virus

Based on the results of cell culture assays, the rAAV-RAB expression vector was tested in vivo in laboratory mice to confirm, safety, efficiency, and durability of antibody production in the central nervous system (CNS) and systemically. Anti-Rabies neutralizing antibodies present in mouse brains, liver and sera demonstrate the effectiveness of antibody gene therapy in this model system and provides essential proof of concept.

Animal Immunization Procedure: C57BL/6J female mice approximately 30 days of age were used by injecting cohorts of 6 mice/dose group, dosed intravenously (IV) or intraperitoneally (IP) with AAV-RAB at 1.5×10¹³ or 6.0×10¹³ vector genomes per kg body weight (vg/kg). The mice weighed approximately 25 g. The mice were bled from the tail vein at 14 and 24 days and necropsied at 30 or 60 days after treatment when serum was collected for antibody assay, and brains and liver tissue were collected to assay for anti-rabies antibodies. Mice were assigned to unvaccinated controls (3 mice/time point) or vaccinates (6 mice/time point) which were injected IV via the tail vein or IP with either high or low dose of AAV-RAB in a total volume of 100 ul. Untreated mice served as controls. Each cohort were necropsied at 30 and 60 days after dosing and were examined for lesions grossly or by histopathology.

Antibody Assays: Anti-rabies antibodies in mouse sera and tissues were assayed for anti-rabies antibodies using Rapid fluorescent focus inhibition test (RFFIT) and immunohistochemical test (Palucha, et al 2005).

Data Collection and Analysis: Data were collected, entered into spread sheets and analyzed statistically with statistical analysis software and graphic display. The experimental design for mouse trials is cross-sectional sampling of un-treated and treated mouse cohorts at monthly intervals from 30 days to 60 days after treatment. Antibody gene therapy dependent effects were evaluated using the one-way ANOVA with Tukey' multiple comparison test for means spread or non-parametric t-test. Significance was set at P<0.05.

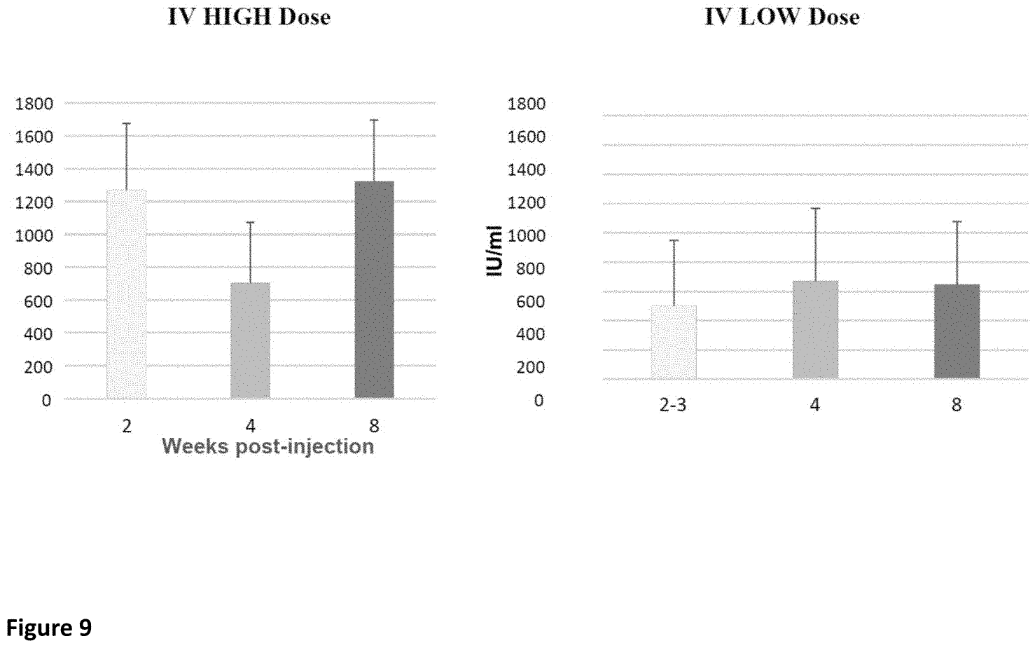

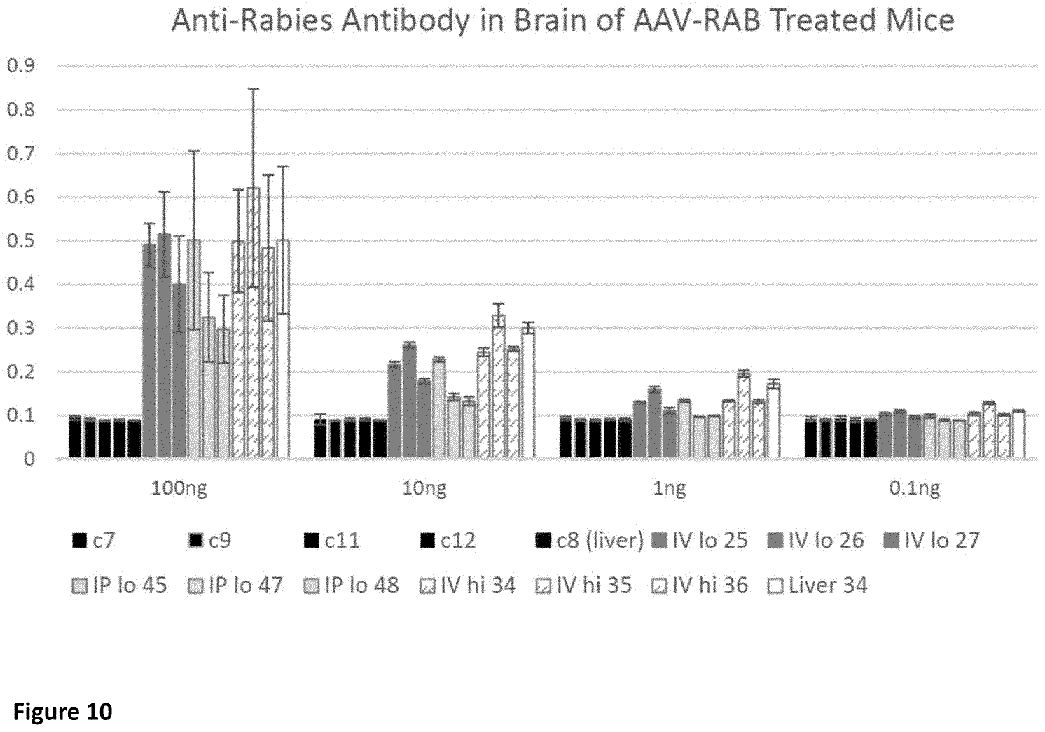

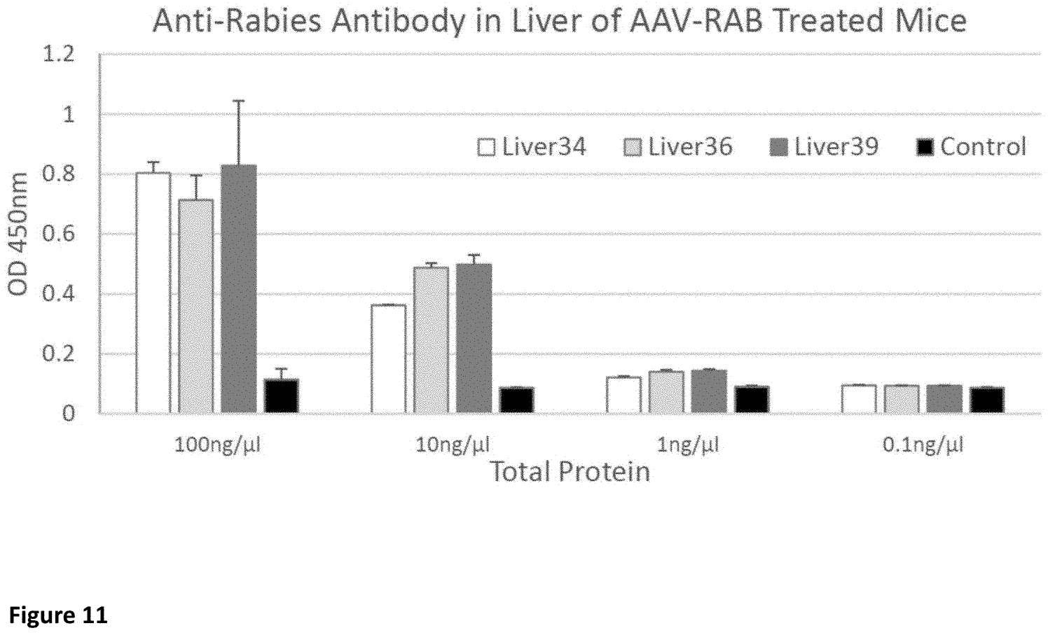

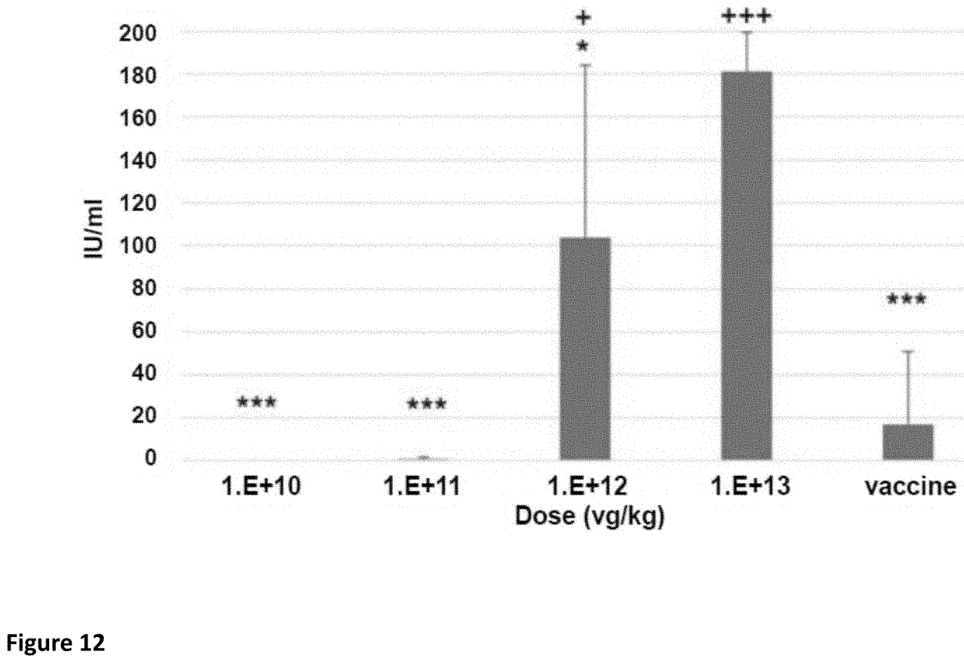

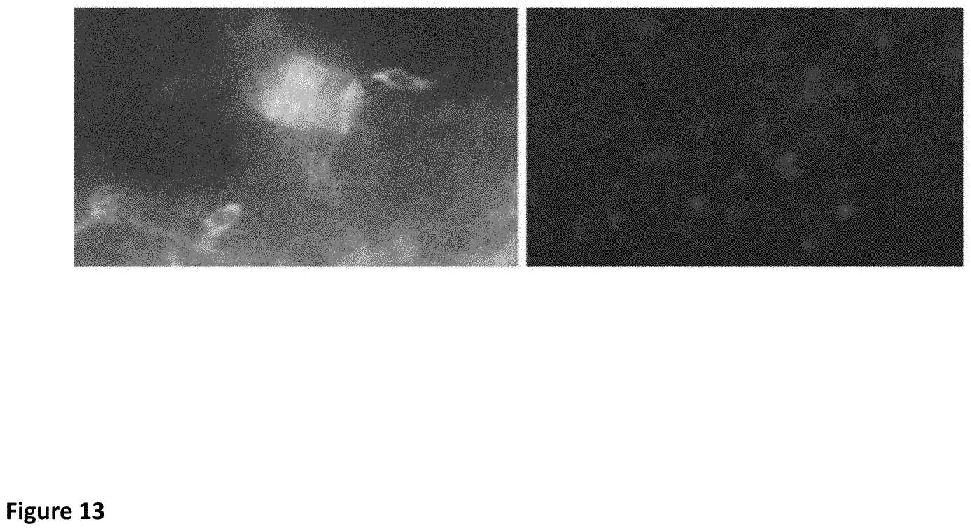

Summary of Results. Mice given a single IV or IP injection of AAV-RAB anti-rabies antibody gene therapy at a dose of 1.5×10¹³ vg/kg produce protective serum rabies virus neutralizing antibody titers that far exceeded titers produced by standard rabies vaccines (hundreds fold greater) and protective antibodies were produced systemically and in the CNS. Results are shown in Tables 2-5 below and in FIGS. 9-13.

TABLE 2
Anti-Rabies Antibody Titers. Interval Tail Bleeding
on Days 11, 14, and 21 Post-Treatment

| | | AAV Dose and | | |
|---|---|---|---|---|
| Mouse No. | Days Post Tr. | Route | Ab Titer | IU/ml |
| 1 | 14 | AAV-IV-High | Insufficient | |
| | | | volume | |
| 2 | 14 | AAV-IV-High | >1:174,692 | >1,519 |
| 3 | 14 | AAV-IV-High | >1:174,692 | >1,519 |
| 4 | 14 | AAV-IV-High | 1:142,857 | 1,242 |
| 5 | 14 | AAV-IV-High | 1:159,750 | 1,389 |
| 6 | 14 | AAV-IV-High | 1:159,750 | 1,389 |
| 7 | 14 | AAV-IV-High | >1:174,692 | >1,519 |
| 8 | 14 | AAV-IV-High | 1:34,800 | 302.6 |
| 9 | 21 | AAV-IV-Low | 1:159,750 | 1,389 |
| 10 | 21 | AAV-IV-Low | 1:34,800 | 302.6 |
| 11 | 21 | AAV-IV-Low | 1:34,800 | 302.6 |
| 12 | 21 | AAV-IV-Low | 1:20,432 | 177.7 |
| 13 | 14 | AAV-IV-Low | 1:59,744 | 520 |
| 14 | 14 | AAV-IV-Low | 1:24,747 | 215 |
| 15 | 14 | AAV-IV-Low | 1:6,300 | 55 |
| 16 | 14 | AAV-IV-Low | 1:123,765 | 1,076 |
| 17 | 14 | AAV-IP-High | >1:174,692 | >1,519 |
| 18 | 14 | AAV-IP-High | 1:67,491 | 587 |
| 19 | 11 | AAV-IP-High | 1:3,125 | 31 |
| 20 | 11 | AAV-IP-High | 1:1,300 | 13 |
| 21 | 14 | AAV-IP-High | 1:34,800 | 303 |
| 22 | 14 | AAV-IP-High | 1:18,086 | 157 |
| 23 | 14 | AAV-IP-Low | 1:102,160 | 888 |
| 24 | 14 | AAV-IP-Low | 1:113,263 | 985 |

Dose designated Low = 1.5 × 10(13) vg/kg
IV = Intravenous tail vein injection
IP = Intraperitoneal injection
> = highest titer tested, actual end-point is higher

TABLE 3
Anti-Rabies Antibody Titers. Necropsy of 30
Day Cohort on Post-Treatment Days 27 and 28

| | | AAV Dose and | | |
|---|---|---|---|---|
| Mouse No. | Days Post Tr. | Route | Ab Titer | IU/ml |
| 1 | 27 | AAV-IV-High | 1:123,735 | 1237 |
| 2 | 27 | AAV-IV-High | 1:26,718 | 267 |
| 3 | 27 | AAV-IV-High | 1:45,687 | 457 |
| 4 | 27 | AAV-IV-High | 1:67,491 | 675 |
| 5 | 27 | AAV-IV-High | 1:67,491 | 675 |
| 6 | 27 | AAV-IV-High | 1:31,950 | 320 |
| 7 | 27 | AAV-IV-High | 1:67,491 | 675 |
| 8 | 27 | AAV-IV-High | 1:133,591 | 1,336 |
| 9 | 28 | AAV-IV-Low | 1:142,857 | 1,429 |
| 10 | 28 | AAV-IV-Low | 1:49,326 | 493 |
| 11 | 28 | AAV-IV-Low | 1:123,735 | 1,237 |
| 12 | 28 | AAV-IV-Low | 1:10,000 | 100 |
| 13 | 27 | AAV-IV-Low | 1:59,744 | 597 |
| 14 | 27 | AAV-IV-Low | 1:5,100 | 51 |
| 15 | 27 | AAV-IV-Low | 1:113,263 | 1,133 |
| 16 | 27 | AAV-IV-Low | 1:33,489 | 335.00 |
| 17 | 27 | AAV-IP-High | 1:59,744 | 597 |
| 18 | 27 | AAV-IP-High | >1:174,692 | >1,747 |
| 19 | 11 | AAV-IP-High | 1:3,125 | 31.25 |
| 20 | 11 | AAV-IP-High | 1:1,300 | 13 |
| 21 | 27 | AAV-IP-High | 1:159,750 | 1,598 |
| 22 | 27 | AAV-IP-High | 1:42,724 | 427 |
| 23 | 27 | AAV-IP-Low | 1:40,269 | 403 |
| 24 | 27 | AAV-IP-Low | 1:151,565 | 1,516 |
| 41 | 27 | AAV-IP-High | 1:90,434 | 904 |
| 42 | 27 | AAV-IP-High | 1:13,500 | 135 |
| 43 | 27 | AAV-IP-High | 1:113,263 | 1,133 |
| 44 | 27 | AAV-IP-High | 1:33,489 | 335 |

Dose designate High = 6.0 × 10(13) vg/kg
Dose designated Low = 1.5 × 10(13) vg/kg
IV = Intravenous tail vein injection
IP = Intraperitoneal injection
> = highest titer tested, actual end-point is higher

TABLE 4
Anti-Rabies Antibody Titers. Interval Tail
Bleeding at 35 days of the 60 day cohort

| | | AAV | | |
|---|---|---|---|---|
| Mouse No. | Days Post Tr. | Dose/Route | Ab Titer | IU/ml |
| 25 | 35 | AAV-IV-Low | 1:90,434 | 786 |
| 26 | 35 | AAV-IV-Low | 1:18,086 | 157 |
| 27 | 35 | AAV-IV-Low | 1:142,857 | 1,242 |
| 28 | 35 | AAV-IV-Low | 1:102,160 | 888 |
| 29 | 35 | AAV-IV-Low | 1:42,724 | 372 |
| 30 | 35 | AAV-IV-Low | 1:45,687 | 397 |
| 31 | 35 | AAV-IV-Low | 1:133,591 | 1,162 |
| 32 | 35 | AAV-IV-Low | 1:20,432 | 178 |
| 33 | 35 | AAV-IV-High | >1:174,692 | >1,519 |
| 34 | 35 | AAV-IV-High | >1:174,692 | >1,519 |
| 35 | 35 | AAV-IV-High | >1:174,692 | >1,519 |
| 36 | 35 | AAV-IV-High | 1:102,160 | 888 |
| 37 | 35 | AAV-IV-High | 1:67,491 | 587 |
| 38 | 35 | AAV-IV-High | >1:174,692 | >1,519 |
| 39 | 35 | AAV-IV-High | >1:174,692 | >1,519 |
| 40 | 35 | AAV-IV-High | >1:174,692 | >1,519 |
| 45 | 35 | AAV-IP-Low | >1:174,692 | >1,519 |
| 46 | 35 | AAV-IP-Low | 1:7,000 | 61 |
| 47 | 35 | AAV-IP-Low | 1:40,269 | 350 |
| 48 | 35 | AAV-IP-Low | 1:10,000 | 87 |

Dose designate High = 6.0 × 10(13) vg/kg
Dose designated Low = 1.5 × 10(13) vg/kg
IV = Intravenous tail vein injection
IP = Intraperitoneal injection
> = highest titer tested, actual end-point is higher

TABLE 5
Anti-Rabies Antibody in Untreated Control Mice

| Mouse No. | Necropsy date | TITER |
|---|---|---|
| C-1 | Apr. 1, 2019 | <1:280 |
| C-2 | Apr. 1, 2019 | <1:280 |
| C-3 | Apr. 11, 2019 | <1:280 |
| C-4 | Apr. 11, 2019 | <1:280 |
| C-5 | Apr. 11, 2019 | <1:280 |
| C-6 | Apr. 11, 2019 | <1:280 |

Titers of <128 are known to equal no anti-rabies antibodies.

In summary, all sera collected at every time point starting with 11 days post-treatment were positive for anti-rabies antibody titers that exceed the minimal protective level by many fold greater than that recommended by the World Health Organization (0.5 IU). At 14 days after treatment the AAV high dose administered IV achieved the highest recorded antibody titer of >1:174,692 (>1,519 IU) in 3 of 8 mice and 4 more mice had titers of 1:142,875 (1,242 IU). Only one of these 8 mice had a titer of 1:34,800 (303 IU) which is 600 time that presumptive minimal protective titer (MPT). All sera from six (6) control, untreated mice were negative for anti-rabies antibodies. Anti-rabies antibody titers remain high 35 and 60 days after treatment. Titers range from 1:1,300 (13 IU, 26×MPT) at 11 days post treatment to >1:174,692 (>1,519 IU, 3,000×MPT) at several time points. Titers vary between time pints, dose and route, but all are many fold greater than a recommended minimal protective serum titer. The route of administration (IV versus IP) and the dose do not seem to result in any meaningful difference in serum titers.

There is some suggestion, without statistical testing, that IV administration and the high dose may result in higher titers, which may in fact be irrelevant since all doses and routes at all time points exceed the protective serum antibody by tens to thousands fold. Data shown here confirm that anti-rabies antibodies are in brain (immunohistochemistry) and liver (ELISA). Brain and liver homogenates are being tested for neutralizing antibody assay.

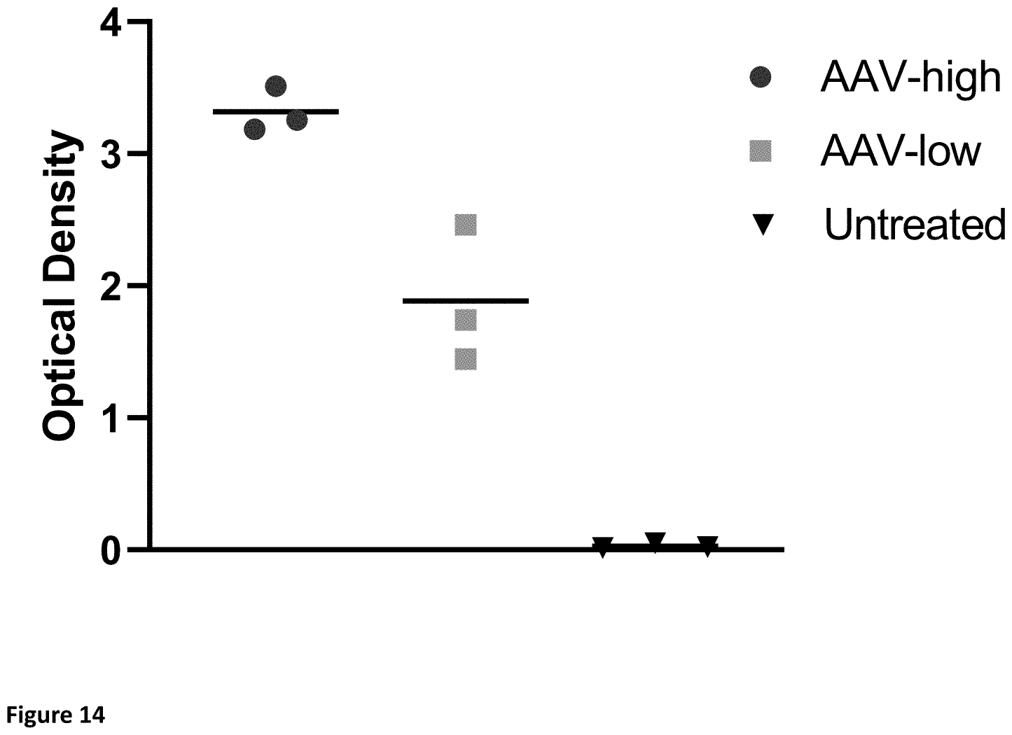

AAV-RAB treatment of cats and dogs is underway. In addition, challenge of mice treated with AAV-RAB with virulent rabies virus in collaboration the Centers for Disease control and Prevention is underway. Expression of anti-rabies antibodies in cats after AAV-RAB treatment is illustrated in FIG. 14.

### Example 4. Minimal Effective Dose of AAV-RAB in Mice Compared with Immunization with a Killed Rabies Virus Vaccine

To assess the lowest AAV-RAB effective dose, C57BL/6J female mice were injected intravenously with 10¹⁰, 10¹¹, 10¹² and 10¹³ viral genomes/kg of body weight. The mice were approximately 8 weeks of age and weighed approximately 25 g. An age-matched cohort of mice were injected subcutaneously with 100 μl of a 1:11 dilution of a commercial killed rabies virus vaccine (IMRAB-3). Cohorts of four (4) mice for each dose and commercial vaccine were necropsied at 30 and 60 days after treatment. At necropsy, mice were exsanguinated to collect serum for neutralizing anti-rabies antibody titers. Brains and livers were collected from each mouse and an aliquot is frozen for neutralizing anti-rabies antibodies and fixed for immunohistochemistry.

### Example 5. Rabies Virus Challenge of Mice Treated with Anti-Rabies Antibody Gene Therapy

We will treat mice with AAV-RAB and then challenge them with virulent rabies virus. This study requires personnel skilled in protection against this lethal human infection and containment facilities capable of isolating personnel and the environment from contamination by rabies virus. The studies will be performed and the Centers for Disease Control and Prevention at Atlanta, Ga.

### REFERENCES

Citation or identification of any document in this application is not an admission that such document is available as prior art to the present invention.
Kaplitt, M. G. et al. Safety and tolerability of gene therapy with an adeno-associated virus (AAV) borne GAD gene for Parkinson's disease: an open label, phase I trial. Lancet 369, 2097-2105 (2007).
Maguire C A, Meijer D H, LeRoy S G, Tierney L A, Brookman M L, Costa F F, Breakefield X O, Stemmer-Rachamimov A, Sena-Esteves M. Preventing growth of brain tumors by creating a zone of resistance. Mol. Ther. (10):1695-702 (2008).
DiFiglia M, Sena-Esteves M, Chase K, Sapp E, Pfister E, Sass M, Yoder J, Reeves P, Pandey R K, Rajeev K G, Manoharan M, Sah D W, Zamore P D, Aronin N. Therapeutic silencing of mutant huntingtin with siRNA attenuates striatal and cortical neuropathology and behavioral deficits. Proc Natl Acad Sci 104(43):17204-9 (20017).
Freskgard P O and Urich E. Antibody therapies in CNS diseases. Neuropharm. 1-18 (2016).
Samulski R J and Muzyczka N: AAV-mediated gene therapy for research and therapeutic Purposes. Ann. Rev. Virol. 1, 427-451 (2014).
Buchlis G, Podsakof G M, Radu A, Hawk S M, Flake A W, Mingozzi F and High K A: Factor IX expression in skeletal muscle of a severe hemophila B Patient 10 years after AAV-mediated gene transfer. Blood. 119, 3038-3041 (2012).
Choudhury S R, Fitzpatrick Z, Harris A F, Maitland S A, Ferreira J S, Zhang Y, Ma S, Sharma R B, Gray-Edwards H L, Johnson J A, Johnson A K, Alonso L C, Punzo C, Wagner K R, Maguire Calif., Kotin R M, Martin D R, Sena-Esteves M. In Vivo Selection Yields AAV-B1 Capsid for Central Nervous System and Muscle Gene Therapy. Mol. Ther. (7):1247-57 (2016).
Foust, K. D. et al. Intravascular AAV9 preferentially targets neonatal neurons and adult astrocytes. Nat. Biotechnol. 27, 59-65 (2009).
Duque S I, Arnold W D, Odermatt P, Ali X Porensky P N, Schmelzer L, Meyer K, Kolb S J, Schumperdi, D, Kaspar B K, Burghes Ah. A large animal model of spinal muscular atrophy and correction of phenotype. Ann. Neurol 77, 399-414 (2015).
Benjamin E Deverman, et al. Cre-dependent selection yields AAV variants for widespread Gene transfer to adult brain. Nat. Biotech. 34 04-209, (2016).
Golebiowski D, van der Bom I M J, Kwon C, Maitland S, Kuhn A L, Bishop N, Curren E, Silva N, Miller A, Westmoreland S, Gounis M, Martin D R, Asaad W & Sena-Esteves M. Safety Study of Intracranial AAVrh8-Mediated Gene Transfer of beta-Hexosaminidase in Non-Human Primates. Mol Ther 21, S149 (2013).
Rabies vaccines, WHO recommendations. Vaccine 28:7140-7142, (2010).
Franka, R, Smith T G, Dyer J L, Wu X, Niezgoda M and Rupprecht C E. Current and future tools for global canine rabies elimination. Antiviral Research. 100, 220-225 (2013).
Mousli M, Turki I, Kharmachi H Saadi M Dellagi K: Recombinant single-chain Fv antibody fragment-alkaline phosphatase conjugate: a novel in vitro tool to estimate rabies viral glycoprotein antigen in vaccine manufacture. J. Viol. Methods, 146 (1-2):246-56, (2007).
McCurdy V J, Johnson A K, Gray-Edwards H L, Randle A N, Brunson B L, Morrison N E, Salibi N, Johnson J A, Hwang M, Beyers R J, Leroy S G, Maitland S, Denney T S, Cox N R, Baker H J, Sena-Esteves M, Martin D R. Sustained normalization of neurological disease after intracranial gene therapy in a feline model. Sci. Trans. Med. (231):231-48 (2014).
Palucha A, Loniewska A, Satheshkumar S, Chachulska A, Umashankar M, Milner M, Haenni A L, Savithri H S. Virus-like particles: models for assembly studies and the design of novel vaccines. Progress in Nucleic Acid Research and Molecular Biology
Klastersky, J and Aoun, M. Opportunistic infection in patients with cancer. Annals of Oncology 15 (2004) 329-335.
Bloch C K, Glaser, C A and Tunkel, Ark. Encephalitis and Myelitis. Infectious Diseases, 4th Ed, Vol 1, 2017, 189-197.
Pieracci, E G, Pearson C M, Wallace R M, et al Vital Signs: Trends in Huan Rabies Deaths and Exposures—United States 1938-2019; Morb. Mortal. Wkl. Rep. (MMWR), 2019, 68: 524-528.
Bourhy, H, Dautry-Varsat, A, Holez, P J, and Salomon J. Rabies, Still Neglected after 125 Years of Vaccination. PLoS Neglected Tropical Diseases. 2010.
Lindenmayer, J M, Wright, J C, Nusbaum, K E, Saville, W J A, Evanson, T and Pappaioanou, M. Reported Rabies Pre-Exposure Immunization of Students at US Colleges of Veterinary Medicine. JVME 40 (3), 2013, 303-309.
Christrian, K A, Blanton, J D, Auslander, M and Rupprecht, C E. Epidemiology of rabies post-exposure prophylaxis—United States of America, 2006-2008. Vaccine 27, 2009, 7156-7161.
Golebiowski D, Bradbury A M, Kwon C S, van der Bom I M J, Stoica L, Johnson A K, Wilson D U, Gray-Edwards H L, Martin D R and Sena-Esteves M. AAV gene therapy strategies for lysosomal storage disorders with central nervous system involvement, in Neuromethods: Gene Delivery and Therapy for Neurological Disorders (Bo X, Verhaagen J, eds.) 98:265-295, 2015.
Rolling et al. “AAV as a viral vector for human gene therapy”, Molecular Biotechnology, vol. 3, 1995, pp 9-15.
Weitzman et al. “Adeno-associated virus (AAV) rep proteins mediate complex-formation between AAV DNA and its integration site in human DNA”, Proceedings of the National Academy of Sciences, vol. 91, No. 13, 1994, pp. 5808-5812.
Walsh et al., “Regulated high-level expression of a human gamma-globin gene introduced into erythroid cells by an adeno-associated vector”, Proceedings of the National Academy of Sciences, vol. 89, No. 15, Aug. 1992, pp. 7257-7261.
Zhou et al. “Adeno-associated virus 2-mediated transduction and erythroid cell-specific expression of a human beta-globin gene” Gene Therapy, vol. 3, Mar. 1996, pp. 223-229.
Gray et al. 2011 Preclinical differences of intravascular AAV9 delivery to neurons and glia: a comparative study of adult mice and nonhuman primates
Lu 2004 Recombinant adeno-associated virus as delivery vector for gene therapy—a review. Stem Cells Dev 2004 February; 13(1):133-45
Gunawardana, S. A and Shaw R. H. Cross-reactive denge virus-derived monoclonal antibodies to Zika virus envelope protein: Panacea or Pandora's box? BMC Infectious Diseases, 2018, pgs. 1-5.
Keeffe, J R, et al. A combination of two human monoclonal antibodies prevents Zika virus escape mutations in non-human primates. Cell Rep. 26, 2018, 1385-94.
Karda, R, et al. Perinatal systemic gene delivery using adeno-associated viral vectors. Frontiers in Molecular Neuroscience, 7, 2014.1-6.
Rahim, A A, et al. In utero administration of Ad5 and AAV pseudotypes to the fetal brain leads to efficient, widespread and tong-term gene expression. Gene Therapy. 19, 2012, 936-46.
Chao, T-Y, et al. SYN023, a novel humanized monoclonal antibody cocktail, for post-exposure prophylaxis of rabies. PLOS Neglected Tropical Diseases. Dec. 20, 2017 https://doi.org/10.1371/journal.pntd.0006133.
Motamed-Gorji, et al. Biological drugs in Gullian-Barre Syndrome: An Update. Current Neuropharmacology. 15, 2017, 938.
Sapparapu G, Fernandez E, Kose N, et al. Neutralizing human antibodies prevent Zika virus replication and fetal disease in mice. Nature 2016; 540: 443-447.
Marston, H, Paula, C, and Fauci, A. Monoclonal Antibodies for Emerging Infectious Diseases. New England Journal of Medicine, 2018.
Mire C E, Geisbert T W. Neutralizing the threat: pan-ebolavirus antibodies close the loop. Trends Mol Med 2017; 23:669-671.
Paules C I, Lakdawala S, McAuliffe J M, et al. The hemagglutinin A stem antibody MEDI8852 prevents and controls disease and limits transmission of pandemic influenza viruses. J Infect Dis 2017; 216:356-365.
Xuan-Yi Wang, Bin Wang, Yu-Mei Wen. (2019) From therapeutic antibodies to immune complex vaccines. npj Vaccines 4:1.
Robert W Cross, Kathryn M Hastie, Chad E Mire, James E Robinson, Thomas W Geisbert, Luis M Branco, Erica Ollmann Saphire, Robert F Garry. (2019) Antibody therapy for Lassa fever. Current Opinion in Virology 37, 97-104.
Andreas H. Laustsen. (2019) How can monoclonal antibodies be harnessed against neglected tropical diseases and other infectious diseases? Expert Opinion on Drug Discovery 40, 1-10.
Nurgun Kose, Julie M. Fox, Gopal Sapparapu, Robin Bombardi, Rashika N. Tennekoon, A. Dharshan de Silva, Sayda M. Elbashir, Matthew A. Theisen, Elisabeth Humphris-Narayanan, Giuseppe Ciaramella, Sunny Himansu, Michael S. Diamond, James E. Crowe. (2019) A lipid-encapsulated mRNA encoding a potently neutralizing human monoclonal antibody protects against chikungunya infection. Science Immunology 4:35, 647.
Joshua Tan, Luca Piccoli, Antonio Lanzavecchia. (2019) The Antibody Response to Plasmodium falciparum: Cues for Vaccine Design and the Discovery of Receptor-Based Antibodies. Annual Review of Immunology 37:1, 225-246.
Giuseppe Stefanetti, Nihal Okan, Avner Fink, Erica Gardner, Dennis L. Kasper. (2019) Glycoconjugate vaccine using a genetically modified O antigen induces protective antibodies to Francisella tularensis. Proceedings of the National Academy of Sciences 116:14, 7062-7070.
Ki Hyun Kim, Jinhee Kim, Meehyun Ko, June Young Chun, Hyori Kim, Seungtaek Kim, Ji-Young Min, Wan Beom Park, Myoung-don Oh, Junho Chung, Florian Krammer. (2019) An anti-Gn glycoprotein antibody from a convalescent patient potently inhibits the infection of severe fever with thrombocytopenia syndrome virus. PLOS Pathogens 15:2, e1007375.
Pelfrene, M. Mura, A. Cavaleiro Sanches, M. Cavaleri. (2019) Monoclonal antibodies as anti-infective products: a promising future? Clinical Microbiology and Infection 25:1, 60-64.
Joseph P. Cornish, Reed F. Johnson. 2019. Swords to Ploughshares and Back: The Continuing Threat of Immunomodulatory Research and Development. Defense Against Biological Attacks, 195-223.
Jose L. Garrido, Joseph Prescott, Mario Calvo, Felipe Bravo, Raymond Alvarez, Alexis Salas, Raul Riquelme, Maria L. Rioseco, Brandi N. Williamson, Elaine Haddock, Heinz Feldmann, Maria I. Barria. (2018) Two recombinant human monoclonal antibodies that protect against lethal Andes hantavirus infection in vivo. Science Translational Medicine 10:468, 6420.
Lisa K. Blum, Julia Z. Adamska, Dale S. Martin, Alison W. Rebman, Serra E. Elliott, Richard R. L. Cao, Monica E. Embers, John N. Aucott, Mark J. Soloski, William H. Robinson. (2018) Robust B Cell Responses Predict Rapid Resolution of Lyme Disease. Frontiers in Immunology 9.
Raffael Nachbagauer, David Shore, Hua Yang, Scott K. Johnson, Jon D. Gabbard, S. Mark Tompkins, Jens Wrammert, Patrick C. Wilson, James Stevens, Rafi Ahmed, Florian Krammer, Ali H. Ellebedy, Stacey Schultz-Cherry. (2018) Broadly Reactive Human Monoclonal Antibodies Elicited following Pandemic H1N1 Influenza Virus Exposure Protect Mice against Highly Pathogenic H5N1 Challenge. Journal of Virology 92:16
Arun K. Dangi, Rajeshwari Sinha, Shailja Dwivedi, Sanjeev K. Gupta, Pratyoosh Shukla. (2018) Cell Line Techniques and Gene Editing Tools for Antibody Production: A Review. Frontiers in Pharmacology 9.

Davide Corti, Fabio Benigni, Daniel Shouval. (2018) Viral envelope-specific antibodies in chronic hepatitis B virus infection. Current Opinion in Virology 30, 48-57.
Centers for Disease Control and Prevention. Recommendations of the Advisory Committee on Immunization Practices (ACIP): diphtheria, tetanus, and pertussis—recommendations for vaccine use and other preventive measures. MMWR Morb Mortal Wkly Rep 1991; 40(RR-10): 1-25.
Giangrasso J, Smith R K. Misuse of tetanus immunoprophylaxis in wound care. Ann Emerg Med 1985; 14: 573-9.
Passen E L, Andersen B R. Clinical tetanus despite a “protective” level of toxin-neutralizing antibody. JAMA 1986; 225(9): 1171-3.
A B Lang, S J Cryz Jr, U Schürch, M T Ganss and U Bruderer. Immunotherapy with human monoclonal antibodies. Fragment A specificity of polyclonal and monoclonal antibodies is crucial for full protection against tetanus toxin. J Immunol, 1993, 151 (1) 466-472
N. Scott, O. Qazi, M. J. Wright, N. F. Fairweather, M. P. Deonarain Characterisation of a panel of anti-tetanus toxin single-chain Fvs reveals cooperative binding. Mol Immunol, 47 (2010), pp. 1931-1941
Ivana Lukic, Aleksandra Inic-anada, Emilija Marinkovic, Radmila Miljkovic, Marijana Stojanovic. Cooperative binding of anti-tetanus toxin monoclonal antibodies: Implications for designing an efficient biclonal preparation to prevent tetanus toxin intoxication. Vaccine, 2018, Pages 3764-3771
Foust, K D, Nurre, E, Montgomery, C L, Hernandez, A, Chan, C M and Kaspar, B K (2009). Intravascular AAV9 preferentially targets neonatal neurons and adult astrocytes. Nat Biotechnol 27: 59-65.
Foust, K D, Wang, X, McGovern, V L, Braun, L, Bevan, A K, Haidet, A M et al. (2010). Rescue of the spinal muscular atrophy phenotype in a mouse model by early postnatal delivery of SMN. Nat Biotechnol 28: 271-274.
Foust, K D, Salazar, D L, Likhite, S, Ferraiuolo, L, Ditsworth, D, Ilieva, H et al. (2013). Therapeutic AAV9-mediated suppression of mutant SOD1 slows disease progression and extends survival in models of inherited ALS. Mol Ther 21: 2148-2159.
Fu, H, Dirosario, J, Killedar, S, Zaraspe, K and McCarty, D M (2011). Correction of neurological disease of mucopolysaccharidosis IIIB in adult mice by rAAV9 trans-blood brain barrier gene delivery. Mol Ther 19: 1025-1033.
Ruzo, A, Marcó, S, Garcia, M, Villacampa, P, Ribera, A, Ayuso, E et al. (2012).
Correction of pathological accumulation of glycosaminoglycans in central nervous system and peripheral tissues of MPSIIIA mice through systemic AAV9 gene transfer. Hum Gene Ther 23: 1237-1246.
Ahmed, S S, Li, H, Cao, C, Sikoglu, E M, Denninger, Ark., Su, Q et al. (2013). A single intravenous rAAV injection as late as P20 achieves efficacious and sustained CNS Gene therapy in Canavan mice. Mol Ther 21: 2136-2147.
Garg, S K, Lioy, D T, Cheval, H, McGann, J C, Bissonnette, J M, Murtha, M J et al. (2013).
Systemic delivery of MeCP2 rescues behavioral and cellular deficits in female mouse models of Rett syndrome. J Neurosci 33: 13612-13620.
Weismann, C M, Ferreira, J, Keeler, A M, Su, Q, Qui, L, Shaffer, S A et al. (2015).
Systemic AAV9 gene transfer in adult GM1 gangliosidosis mice reduces lysosomal storage in CNS and extends lifespan. Hum Mot Genet 24: 4353-4364.
Yang, B, Li, S, Wang, H, Guo, Y, Gessler, D J, Cao, C et al. (2014). Global CNS transduction of adult mice by intravenously delivered rAAVrh.8 and rAAVrh.10 and nonhuman primates by rAAVrh.10. Mol Ther 22: 1299-1309.
Duque, S, Joussemet, B, Riviere, C, Marais, T, Dubreil, L, Douar, A M et al. (2009). Intravenous administration of self-complementary AAV9 enables transgene delivery to adult motor neurons. Mol Ther 17: 1187-1196.
Bevan, A K, Duque, S, Foust, K D, Morales, P R, Braun, L, Schmelzer, L et al. (2011). Systemic gene delivery in large species for targeting spinal cord, brain, and peripheral tissues for pediatric disorders. Mol Ther 19: 1971-1980.
Gombash, S E, Cowley, C J, Fitzgerald, J A, Hall, J C, Mueller, C, Christofi, F L et al. (2014). Intravenous AAV9 efficiently transduces myenteric neurons in neonate and juvenile mice. Front Mol Neurosci 7: 81.
Benskey, M J, Kuhn, N C, Galligan, J J, Garcia, J, Boye, S E, Hauswirth, W W et al. (2015). Targeted gene delivery to the enteric nervous system using AAV: a comparison across serotypes and capsid mutants. Mol Ther 23: 488-500.
DiMattia, M A, Nam, H J, Van Vliet, K, Mitchell, M, Bennett, A, Gurda, B L et al. (2012). Structural insight into the unique properties of adeno-associated virus serotype 9. J Virol 86: 6947-6958.
Sourav R Choudhury, Zachary Fitzpatrick, Anne F Harris, Stacy A Maitland, Jennifer S Ferreira, Yuanfan Zhang, Shan Ma, Rohit B Sharma, Heather L Gray-Edwards, Jacob A Johnson, Aime K Johnson, Laura C Alonso, Claudio Punzo, Kathryn R Wagner, Casey A Maguire, Robert M Kotin, Douglas R Martin and Miguel Sena-Esteves. In Vivo Selection Yields AAV-B1 Capsid for Central Nervous System and Muscle Gene Therapy. Molecular Therapy (2016) 24, 1247.
Sourav R Choudhury, Anne F Harris, Damien Cabral, Allison Keeler, Ellen Sapp, Jennifer S Ferreira, Heather L Gray-Edwards, Jacob A Johnson, Aime K Johnson, Qin Su, Lorelei Stoica, Marian DiFiglia, Neil Aronin, Douglas R Martin, Guanqping Gao and Miguel Sena-Esteves.
Widespread Central Nervous System Gene Transfer and Silencing After Systemic Delivery of Novel AAV-AS Vector. (2016) Molecular Therapy. 24, 726.
Tzu-Yuan Chao, et al. SYN023 a novel humanized monoclonal antibody cocktail for post-exposure prophylaxis of rabies. Neglected Tropical Diseases, 2017
Franks, Richard, et al. Rabies virus pathogenesis in relationship to intervention with inactivated and attenuated rabies vaccines. Vaccine, 27 (2009), 7149.
Rahim, A A. et al. In utero administration of AD5 and AAV pseudotypes to the fetal brain leads to efficient, widespread and long-term gene expression. Gene Therapy, 19 (2012), 936.
De Kruif, John, et al. A human monoclonal antibody cocktail as a novel component of rabies postexposure prophylaxis. Annu. Rev. Med. 2007, 58, 359.
Keefe, J R, et al. A combination of two human monoclonal antibodies prevents zika virus escape mutations in non-human primates. Cell Rep. 25, 2018, 1385.
Dietzschold, B., M. Gore, P. Casali, Y. Ueki, C. E. Rupprecht, A. L. Notkins, and H. Koprowski. 1990. Biological characterization of human monoclonal antibodies to rabies virus. J. Virol. 64:3087-3090. 7
Dietzschold, B., M. Tollis, M. Lafon, W. H. Wunner, and H. Koprowski. 1987. Mechanisms of rabies virus neutralization by glycoprotein-specific monoclonal antibodies. Virology 161:29-36.
Enssle, K., R. Kurrle, R. Kohler, H. Muller, E. J. Kanzy, J. Hilfenhaus, and F. R. Seiler. 1991. A rabies-specific human monoclonal antibody that protects mice against lethal rabies. Hybridoma 10:547-556.
Flamand, A., H. Raux, Y. Gaudin, and R. W. Ruigrok. 1993. Mechanisms of rabies virus neutralization. Virology 194:302-313. 12. Hanlon, C. A., C. A. DeMattos, C. C. DeMattos, M. Niezgoda, D. C. Hooper, H. Koprowski, A. Notkins, and C. E. Rupprecht. 2001. Experimental utility of rabies virus-neutralizing human monoclonal antibodies in post-exposure prophylaxis. Vaccine 19:3834-3842.
Hanlon, C. A., M. Niezgoda, P. A. Morrill, and C. E. Rupprecht. 2001. The incurable wound revisited: progress in human rabies prevention? Vaccine 19:2273-2279.
Hanlon, C. A., M. Niezgoda, and C. E. Rupprecht. 2002. Postexposure prophylaxis for prevention of rabies in dogs. Am. J. Vet. Res. 63:1096-2100.
Irie, T., and A. Kawai. 2002. Studies on the different conditions for rabies virus neutralization by monoclonal antibodies #1-46-12 and #7-1-9. J. Gen. Virol. 83:3045-3053.
Marissen, W. E., R. A. Kramer, A. Rice, W. C. Weldon, M. Niezgoda, M. Faber, J. W. Slootstra, R. H. Meloen, M. Clijsters-van der Horst, T. J. Visser, M. Jongeneelen, S. Thijsse, M. Throsby, J. de Kruif, C. E. Rupprecht, B. Dietzschold, J. Goudsmit, and A. B. H. Bakker. 2005. Novel rabies neutralizing epitope recognized by human monoclonal antibody: fine mapping and escape mutant analysis. J. Virol. 79:4672-4678.
Montano-Hirose, J. A., M. Lafage, P. Weber, H. Badrane, N. Tordo, and M. Lafon. 1993. Protective activity of a murine monoclonal antibody against European bat lyssavirus 1 (EBL1) infection in mice. Vaccine 11:1259-1266.
Prosniak, M., M. Faber, C. A. Hanlon, C. E. Rupprecht, D. C. Hooper, and B. Dietzschold. 2003. Development of a cocktail of recombinant-expressed human rabies virus-neutralizing monoclonal antibodies for postexposure prophylaxis of rabies. J. Infect. Dis. 188:53-56.
Bakker, A B H, et al Novel human monoclonal antibody combination effectively neutralizing natural rabies virus variant and individual in vitro escape mutants. J. Viol. 79, 2005, 9062-68.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating and/or preventing an infection by rabies lyssavirus in a subject in need thereof, the method comprising administering to the subject a dose of an adeno-associated virus (AAV) vector that expresses in the subject an immunoglobulin that binds and neutralizes rabies lyssavirus.

2. The method of claim 1, wherein the method is effective for treating rabies lyssavirus encephalitis.

3. The method of claim 1, wherein the AAV vector expresses systemically in the subject an immunoglobulin that binds and neutralizes rabies lyssavirus.

4. The method of claim 1, wherein the AAV vector expresses in the nervous system of the subject an immunoglobulin that binds and neutralized rabies lyssavirus.

5. The method of claim 4, wherein the AAV vector expresses in the central nervous system and/or in the peripheral nervous system of the subject an immunoglobulin that binds and neutralized rabies lyssavirus.

6. The method of claim 1, wherein the AAV vector is administered via a route selected from the group consisting of intravenously, intramuscularly, subcutaneously, orally, or nasally.

7. The method of claim 1, wherein the immunoglobulin binds Glycoprotein G of rabies lyssavirus.

8. The method of claim 1, wherein the immunoglobulin is an antibody or an antibody fragment selected from the group consisting of monoclonal antibodies, camelid antibodies, single domain antibodies (sdAb), intracellular antibodies, recombinant antibodies, multispecific antibodies, Fv, Fab, F(ab)₂, F(ab)₃, Fab′, Fab′-SH, F(ab′)₂, single chain variable fragment antibodies (scFv), di-scFv, Fc, pFc′, and scFvFc.

9. The method of claim 1, wherein the AAV vector is administered to the subject at a dose of no more than about 10¹³, 10¹², 10¹¹, 10¹⁰, 10⁹, 10⁸, 10⁷, 10⁶, 10⁵, or 10⁴ viral genomes (vg)/kg body weight of the subject.

10. The method of claim 1, wherein the dose of the AAV vector is effective for expressing the immunoglobulin in the subject systemically at a titer of at least about 1:100, 1:500, 1:10000, 1:50000, or 1:100000, (or at a concentration of at least about 0.5 IU/ml, 1 IU/ml, 5 IU/ml, 10 IU/ml, 50 IU/ml, 100 IU/ml, 500 IU/ml, or 1000 IU/ml).

11. The method of claim 1, wherein the dose of the AAV vector is effective for expressing the immunoglobulin in the nervous system of the subject, optionally the central nervous system of the subject or the peripheral nervous system of the subject, at a titer of at least about 1:100, 1:500, 1:10000, 1:50000, or 1:100000, (or at a concentration of at least about 0.5 IU/ml, 1 IU/ml, 5 IU/ml, 10 IU/ml, 50 IU/ml, 100 IU/ml, 500 IU/ml, or 1000 IU/ml).

12. The method of claim 1, wherein the dose of the AAV vector is effective for expressing the immunoglobulin in the subject systemically at a titer of at least about 1:100, 1:500, 1:10000, 1:50000, or 1:100000, (or at a concentration of at least about 0.5 IU/ml, 1 IU/ml, 5 IU/ml, 10 IU/ml, 50 IU/ml, 100 IU/ml, 500 IU/ml, or 1000 IU/ml) as soon as 1, 2, 4, 6, 8, 10, 12, 14, 16, 18, 20, 22, 24, 36, 48, 60, 72, 96, 120, 144, 168, 192, 216, 240, or 256 hours after treatment.

13. The method of claim 1, wherein the dose of the AAV vector is effective for expressing the immunoglobulin in the nervous system of the subject, optionally the central nervous system of the subject or the peripheral nervous system of the subject, at a titer of at least about 1:100, 1:500, 1:10000, 1:50000, or 1:100000, (or at a concentration of at least about 0.5 IU/ml, 1 IU/ml, 5 IU/ml, 10 IU/ml, 50 IU/ml, 100 IU/ml, 500 IU/ml, or 1000 IU/ml) as soon as 1, 2, 4, 6, 8, 10, 12, 14, 16, 18, 20, 22, 24, 36, 48, 60, 72, 96, 120, 144, 168, 192, 216, 240, or 256 hours after treatment.

14. The method of claim 1, consisting of administering a single dose of the adeno-associated virus (AAV) vector that expresses in the subject an immunoglobulin that binds and neutralizes rabies lyssavirus.

15. The method of claim 1, wherein the subject expresses the immunoglobulin at a titer of at least about 1:100, 1:500, 1:10000, 1:50000, or 1:100000, (or at a concentration of at least about 0.5 IU/ml, 1 IU/ml, 5 IU/ml, 10 IU/ml, 50 IU/ml, 100 IU/ml, 500 IU/ml, or 1000 IU/ml) as long as 1, 2, 4, 6, 8, 10, 12, 14, 16, 18, or 20 years after treatment.

16. The method of claim 1, wherein the subject is immunocompromised.

17. The method of claim 1, wherein the subject is a newborn infant.

18. The method of claim 1, wherein the subject is human subject.

19. The method of claim 1, wherein the subject is an animal subject, optionally a companion animal such as a dog or cat.

20. An adeno-associated virus (AAV) vector that expresses in the central nervous system (CNS) of the subject an immunoglobulin that binds and neutralizes rabies lyssavirus.

21. The vector of claim 20, wherein the immunoglobulin binds Glycoprotein G of rabies lyssavirus.

22. The vector of claim 20, wherein the immunoglobulin is an antibody or an antibody fragment selected from the group consisting of monoclonal antibodies, camelid antibodies, single domain antibodies (sdAb), intracellular antibodies, recombinant antibodies, multispecific antibodies, Fv, Fab, F(ab)₂, F(ab)₃, Fab′, Fab′-SH, F(ab′)₂, single chain variable fragment antibodies (scFv), di-scFv, Fc, pFc′, and scFvFc.

23. The vector of claim 20, wherein the vector expresses a heavy chain of the immunoglobulin via a single promoter and the vector expresses a light chain of the immunoglobulin via exon skipping induced by furin cleavage.

24. The vector of claim 20, wherein the immunoglobulin is a human monoclonal antibody or a fragment thereof.

25. A pharmaceutical composition comprising the vector of claim 20 and a suitable pharmaceutical excipient.

26. The pharmaceutical composition of claim 25, wherein the composition is lyophilized.

27. A method for treating and/or preventing an infection by rabies lyssavirus in a subject in need thereof, the method comprising administering to the subject a dose of the pharmaceutical composition of claim 25.

28. A method for treating and/or preventing an infection by rabies lyssavirus in a subject in need thereof, the method comprising adding an aqueous solution to the pharmaceutical composition of claim 26 to prepare a pharmaceutical solution and administering to the subject a dose of the pharmaceutical solution.

29. A kit comprising as components: (i) the pharmaceutical composition of claim 25; and optionally (ii) a device for administering the pharmaceutical composition to a subject in need thereof.


SEQUENCE LISTING (4 sequences)

SEQ ID NO: 1 - DNA, 1500 bp, Artificial Sequence
  Synthetic- anti-rabies antibody gene pAAV-CBA- CR57-nHL-wpre Clone 2969
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttaaaaactagtcagccaccatggcctgccccggcttcctgtgggccctggtgatcagcacctgcctggaattcagcatggcccaggtgcagctggtgcagagcggagccgaggtgaagaagcccggcagcagcgtgaaggtgagctgcaaggccagcggcggcaccttcaacaggtacaccgtgaactgggtgagacaggccccaggccagggcctggagtggatgggcggcatcatccctatcttcggcaccgccaactacgcccagagattccagggcaggctcaccatcaccgccgacgagagcaccagcaccgcctacatggagctgagcagcctgagaagcgatgacaccgccgtgtac

SEQ ID NO: 2 - DNA, 1500 bp, Artificial Sequence
  Synthetic- anti-rabies antibody gene pAAV-CBA- CR57v1.1-wpre Clone 2970_2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttaaaaactagtcagccaccatggcgacgggttcaagaacttccctacttcttgcatttggcctgctttgtttgccgtggttacaggagggctcggcacaggtgcagctggtgcagagcggagccgaggtgaagaagcccggcagcagcgtgaaggtgagctgcaaggccagcggcggcaccttcaacaggtacaccgtgaactgggtgagacaggccccaggccagggcctggagtggatgggcggcatcatccctatcttcggcaccgccaactacgcccagagattccagggcaggctcaccatcaccgccgacgagagcaccagcaccgcctacatggagctgagcagcctgagaagcgat

SEQ ID NO: 3 - DNA, 1500 bp, Artificial Sequence; identical to SEQ ID NO: 2
  Synthetic- anti-rabies antibody gene pAAV-CBA- CR57v3.1-wpre Clone 2971

SEQ ID NO: 4 - protein, 755 aa, Artificial Sequence
  Synthetic- expression cassette for rabies virus antibody gene
MATGSRTSLLLAFGLLCLPWLQEGSAVKLQQSGGGLVQPGGSLKLSCAASGFDFSRYWMSWVRQAPGKGLEWIGEINPDSSTINYTPSLKDTFIISRDNAKNTLYLQMSKVRSEDTALYYCARQGYDGYHWYFDVWGQGTTVTVSSESLDLAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKRKRRSGSGAPVKQTLNFDLLKLAGDVESNPGPMATGSRTSLLLAFGLLCLPWLQEGSADIQLTQSPSSLSASVGETVTLTCGASENIYGGLNWYQRKQGKSPQLLIYGATKLADGMSSRFSGGGSGRQYFLKITSLHPDDVATYYCQNVLSNPLTFGAGTKLELKRADAAPTVSADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC